(19) United States
(12) Patent Application Publication
(10) Pub. No.: US 2025/0017746 A1
(43) Pub. Date: Jan. 16, 2025

(54) AN AUTOMATED HAND

(71) Applicant: 5TH ELEMENT LIMITED, Christchurch (NZ)

(72) Inventors: Aidan Robert BURGESS, Christchurch (NZ); Cameron Rhys Gwyn RICHARDS, Christchurch (NZ); Darryl John BEST, Diamond Harbour (NZ); David Neil LOVEGROVE, Christchurch (NZ); Ross Hughan DAWSON, Christchurch (NZ); Michael James van der Werff, Christchurch (NZ); Matthew Sultan YAN, Christchurch (NZ)

(73) Assignee: 5TH ELEMENT LIMITED, Christchurch (NZ)

(21) Appl. No.: 18/706,086

(22) PCT Filed: Nov. 1, 2022
(86) PCT No.: PCT/NZ2022/050135

§ 371 Date: Apr. 30, 2024

(30) Foreign Application Priority Data
Nov. 2, 2021 (NZ) 781940

(51) Int. Cl. A61F 2/58; A61F 2/50; A61F 2/68

(52) U.S. Cl. CPC A61F 2/583; A61F 2/68; A61F 2002/5001

(57) ABSTRACT

An automated hand having an arrangement for mounting a digit connector such that the connector can rotate when a force is applied to a digit. The arrangement can include a rigid mount and a resiliently deformable sleeve, with the connector within the sleeve. The automated hand can be compatible with a touchscreen. The automated hand can have a worm drive for rotating a digit, the worm drive including a bearing configured to resist axial loads in both directions. The automated hand can have a thumb with an auxiliary support. A catch can be provided to restrict pivoting of a digit under load. A wrist can be clamped to the hand, can be releasably electrically coupled to the hand, can be locked against rotation, and can include resiliently deformable pieces. A cover can also be provided for an automated hand. The cover can include a knitted body and a structural brace.

### FIELD

This invention relates to an automated hand. The invention also relates to a cover for an automated hand. The invention also relates to a wrist for an automated hand. The invention also relates to a wrist joint. The invention also relates to an assembly including a wrist joint and an automated hand.

### BACKGROUND

Automated hands are commonly used as prosthetic hands, which may be used to grip objects, shake the hand of another person, and perform other tasks commonly carried out by human hands.

### SUMMARY

According to one example there is provided an automated hand comprising:
a. a palm;
b. a rigid mount in or on the palm;
c. a resiliently deformable sleeve located within the rigid mount, the resiliently deformable sleeve having a sleeve aperture therein; and
d. a connector located within the sleeve aperture, the connector having a digit extending therefrom, wherein the digit is moveable relative to the palm; the automated hand being arranged such that the connector can rotate with respect to the mount when a force is applied to the digit extending therefrom.

In some examples, the arrangement is such that each connector can rotate in the plane of the palm.

In some examples, the arrangement is such that each connector can rotate in a plane normal to the plane of the palm.

In some examples, the arrangement is such that each connector can rotate about its longitudinal axis.

In some examples, the arrangement is such that each connector can translate when a force is applied to the digit extending therefrom.

In some examples, the arrangement is such that each connector can translate with three translational degrees of freedom.

In some examples, the automated hand is configured to absorb shocks applied longitudinally to the digits.

In some examples, the connector is part of a digit drive that drives flexion and/or extension of the digit.

In some examples, the connector comprises an actuator.

In some examples, the actuator comprises a motor.

In some examples, the connector is connected between an actuator and the digit.

In some examples, the automated hand comprises an actuator in the digit, the actuator driving flexion and/or extension of the digit.

In some examples, the actuator comprises a pivot between the connector and the rigid mount, the connector being rotatable with respect to the mount about the pivot.

In some examples, the pivot comprises one or more pairs of bearing surfaces, one of the bearing surfaces being on or coupled to the rigid mount and the other being on or coupled to the connector, wherein the bearing surfaces of each pair are in close proximity to each other.

In some examples, the resiliently deformable sleeve has one or more apertures formed therein and wherein one of the bearing surfaces of each pair is on a protrusion that protrudes at least partly through a respective one of the apertures.

In some examples, the automated hand further comprises a rigid sleeve between the connector and the resiliently deformable sleeve.

In some examples, the rigid sleeve is configured to couple the connector to the rigid mount.

In some examples, the rigid sleeve comprises one or more twist-lock features to twist lock to complementary twist lock features on a retainer that retains it with respect to the rigid mount.

In some examples, one of the bearing surfaces of each pair is provided on the rigid sleeve.

In some examples, the automated hand further comprises a seal between the connector and the rigid mount to prevent liquid from entering a sealed region within the automated hand.

In some examples, the seal is located near the pivot.

In some examples, the automated hand comprises:
one or more further rigid mounts attached to the palm;
one or more further resiliently deformable sleeves, each with a respective sleeve aperture therein, wherein each further resiliently deformable sleeve is located within a respective one of the one or more further rigid mounts; and
one or more further connectors, each located within a respective sleeve aperture, each further connector having a further digit extending therefrom;
wherein each further connector can rotate with respect to its respective rigid mount when a force is applied to the further digit extending therefrom.

In some examples, the rigid mounts are integral with each other.

In some examples, the rigid mounts are separate from each other.

In some examples, the rigid mount is configured to provide limits to the rotation of the connector with respect to the mount.

In some examples, an inner surface of the rigid mount within which the resiliently deformable sleeve sits is dimensioned to control the maximum lateral rotation of the connector about one or more axes.

In some examples, the automated hand further comprises a barrier around a portion of the connector that is not within the rigid mount, the barrier configured to limit lateral rotation of the connector about one or more axes.

In some examples, an inner surface of the rigid mount includes one or more rotation restraints to limit rotation of the connector about its longitudinal axis.

In some examples, each connector is coupled to the respective digit by an articulated joint.

In some examples, the resiliently deformable sleeve comprises an elastomer, rubber, silicone, or a polymer.

In some examples, the resiliently deformable sleeve comprises polyurethane or a hydrocarbon-, fluorocarbon- or silica-based elastomer.

In some examples, the resiliently deformable sleeve is a thermoset elastomer.

In some examples, the resiliently deformable sleeve is a thermoplastic material, such as a thermoplastic elastomer.

In some examples, the resiliently deformable sleeve is a thermoset rubber.

In some examples, the resiliently deformable sleeve comprises a foamed composition of one or more of the materials recited in the preceding paragraphs.

In some examples, the resiliently deformable sleeve comprises an alloy or blend of two or more of the materials recited in the preceding paragraphs.

In some examples, the resiliently deformable sleeve comprises a material with a DMTA damping factor of between about 0.05 and about 0.8 over a temperature range of about −20° C. to about 100° C.

In some examples, the resiliently deformable sleeve comprises a material with a DMTA damping factor of between about 0.05 and about 0.5 over a temperature range of about −20° C. to about 100° C.

In some examples, the automated hand comprises a material having a resilience of between about 20% and about 60%.

In some examples, the resiliently deformable sleeve comprises a material having a Shore A hardness of between about 10 and about 90.

In some examples, the resiliently deformable sleeve comprises a material having a Shore A hardness of between about 30 and about 60.

In some examples, the resiliently deformable sleeve comprises a material with a Shore A hardness of about 30.

In some examples, the resiliently deformable sleeve comprises a material having a Shore D hardness of between about 40 and about 90.

According to another example there is provided a touchscreen-compatible automated hand comprising:
a. a conductive member;
b. a digit; and
c. one or more conductive attachments;
wherein the one or more conductive attachments are configured to be attached to the digit to provide a conductive path from the exterior of the digit to the conductive member; and
wherein the automated hand is configured to allow operation of the touchscreen when the conductive path is electrically insulated from the body of a user of the automated hand.

According to another example there is provided a touchscreen-compatible automated hand comprising:
a. a conductive member;
b. a digit; and
c. one or more conductive attachments;
wherein the one or more conductive attachments are configured to be attached to the digit to provide a conductive path from the exterior of the digit to the conductive member, the conductive path being insulated from the body of a user of the automated hand in use.

In some examples, one or more of the conductive attachments comprises a polymer.

In some examples, the polymer is silicone.

In some examples, the one or more of the conductive attachments also comprises a conductive carbon additive.

In some examples, the conductive carbon additive comprises carbon nanotubes.

In some examples, the conductive member is a structural member of the automated hand.

In some examples, the conductive member is a structural member of the digit.

In some examples, the conductive member is part of a linkage of the digit.

In some examples, the one or more conductive attachments comprise a pad and link piece, the pad being located at the exterior of the digit in use and the link piece being in contact with the pad and the conductive member in use.

In some examples, the pad is the conductive attachment defined in any one of the preceding paragraphs.

In some examples, the link piece is a spring that is biased towards contact with the conductive member.

In some examples, the one or more conductive attachments comprise a conductive distal phalanx of the digit.

In some examples, the conductive member is mechanically coupled to the conductive distal phalanx.

In some examples, the conductive path is insulated from terminals of an actuator of the automated hand.

In some examples, the conductive path is insulated from a housing of the actuator.

In some examples, the conductive member and the one or more conductive attachments are configured to, upon one of the conductive attachments touching the touchscreen, cause a touch-sensing array in the touchscreen to sense a change in capacitance within a range indicative of a natural human finger touching the touchscreen.

In some examples, the conductive path terminates at the conductive member.

In some examples, the one or more conductive attachments are configured to be retrofit to an existing, non-touchscreen-compatible automated hand.

According to another example there is provided a method comprising:
attaching one or more conductive attachments to a digit of an automated hand; and
placing one of the one or more conductive attachments in contact with a conductive member of the automated hand;
   wherein the one or more conductive attachments provide a conductive path from an exterior of the digit to the conductive member.

In some examples, the conductive member is a structural member of the automated hand.

In some examples, the conductive member is part of a linkage of the digit.

In some examples, one of the conductive attachments comprises a polymer.

In some examples, the one of the conductive attachments also comprises a conductive carbon additive.

In some examples, the conductive carbon additive comprises carbon nanotubes.

In some examples, attaching one or more conductive attachments to the digit comprises:
attaching a link piece to the digit; and
attaching a pad at the exterior of the digit and in contact with the link piece;
   and wherein placing one of the one or more conductive attachments in contact with a conductive member of the automated hand comprises biasing the link piece towards contact with the conductive member.

In some examples, the method includes retrofitting the one or more conductive attachments to a non-touchscreen-compatible automated hand to produce a touchscreen-compatible automated hand.

In some examples, the method further comprises removing a non-conductive part of the non-touchscreen-compatible automated hand and replacing it with one or more of the conductive attachments.

According to another example there is provided an automated hand comprising:
a. a palm;
b. a digit extending from the palm; and
c. a worm drive configured to rotate the digit relative to the palm in use,
wherein the worm drive includes a worm and a bearing configured to resist axial forces generated by the worm in both directions along the worm longitudinal axis.

In some examples, the bearing is placed between the worm and a motor that drives rotation of the worm.

In some examples, the bearing is a deep groove ball bearing.

In some examples, the worm is restrained against axial movement relative to the bearing.

In some examples, the automated hand comprises flanges secured to the worm, with one flange at each side of the bearing.

In some examples, one or more of the flanges are welded to the worm.

In some examples, the automated hand further comprises a two-part housing, wherein an outer race of the bearing is held between two parts of the housing.

In some examples, the worm drive further comprises a second bearing at the opposite side of the worm from the bearing.

In some examples, the second bearing is smaller than the bearing.

In some examples, the diameter of the second bearing is about two thirds of the diameter of the bearing or less.

In some examples, the diameter of the second bearing is about one half of the diameter of the bearing.

In some examples, the second bearing is configured to resist radial forces on the worm.

In some examples, the second bearing is configured to provide substantially no resistance to the axial forces generated by the worm.

In some examples, the second bearing is configured to be slidable with respect to the longitudinal axis of the worm.

According to another example there is provided a cover for an automated hand, the cover comprising:
a. a cover body including a knitted material; and
b. a hand coupling on the cover body, the hand coupling configured to be secured to an automated hand.

In some examples, the knitted material is configured to have a low resistance to stretching over a first stretch range and a high resistance to stretching over a second stretch range, where the second range is higher than the first range.

In some examples, the resistance to stretching increases sharply between the first stretch range and the second stretch range.

In some examples, the cover body is configured to cover an articulated joint of the automated hand.

In some examples, the cover body is configured to cover a thumb joint of the automated hand.

In some examples, the cover body is configured to cover a joint between the automated hand and a wrist.

In some examples, the cover body is configured to maintain a substantially smooth surface during movement at the joint.

In some examples, the cover is configured to allow movement at the articulated joint over its full range of motion without significantly loading an actuator that drives movement at the articulated joint.

In some examples, the cover is configured to not back drive an actuator that drives movement at the articulated joint when stretched.

In some examples, the knitted material is knitted from elastic thread.

In some examples, the hand coupling is a shaped body having greater rigidity than the cover body material.

In some examples, the hand coupling is made of moulded plastic.

In some examples, the hand coupling at least partly encircles an edge of the cover.

In some examples, the hand coupling is configured to couple to the palm of the automated hand.

In some examples, the cover further comprises a wrist coupling configured to couple to a wrist that the automated hand is coupled to.

In some examples, the cover further comprises a thumb coupling configured to couple to a thumb of the automated hand.

In some examples, the hand coupling is configured to be sandwiched between shell pieces of the automated hand.

In some examples, the wrist coupling is configured to be retained in a groove that encircles the wrist.

In some examples, the cover further comprises one or more additional bodies of a material having greater rigidity than the cover body material to help maintain the shape of the cover.

In some examples, the additional bodies comprise one or more hoops that fully or mostly encircle a part of the cover.

In some examples, the additional bodies are made of moulded plastic.

In some examples, the cover body is configured to allow water to pass through it such that water can drain from a region between the automated hand and the cover.

In some examples, the cover body material is knitted with a sufficiently coarse knit to allow water to pass through the material.

In some examples, the cover body is formed by a 3D knitting process.

In some examples, the cover body comprises a plurality of regions having different properties from each other.

In some examples, the cover body comprises a thumb region that covers at least part of a thumb of the automated hand and a palm region that covers at least part of a palm of the automated hand.

In some examples, two or more of the regions have different stretch characteristics from each other.

In some examples, the thumb region is formed of a less stretchy material than the palm region.

In some examples, two or more of the regions have different coarsenesses from each other.

In some examples, the thumb region is formed of a coarser material than the material of the palm region.

In some examples, the plurality of regions includes a region that is not made of a knitted material.

In some examples, the thumb region is made of a knitted material and the palm region includes a woven material.

In some examples, the cover body has different stretch characteristics in different directions.

In some examples, the cover is configured to approximate the shape of the part of a natural human hand that corresponds to the part of the automated hand that is covered by the cover.

In some examples, the cover body is substantially free of corrugations in use.

In some examples, the cover further comprises one or more reinforced regions configured to lie over protruding features or user input features of the automated hand.

In some examples, the cover further comprises one or more visual indicators configured to lie over user input features of the automated hand.

In some examples, the cover further comprises one or more seams, wherein the seams are sewn, knitted, glued, or joined using bonding tape.

According to another example there is provided a cover for an automated hand, the cover comprising:
a. a cover body including a fabric material; and
b. a structural brace attached to the cover body and configured to support the cover body against external forces;
wherein the cover is configured to be secured to an automated hand.

In some examples, the structural brace is configured to support the cover body against collapse due to gravity.

In some examples, the cover body is configured to cover an articulated joint of the automated hand.

In some examples, the cover body is configured to cover a thumb joint of the automated hand.

In some examples, the cover body is configured to cover a joint between the automated hand and a wrist.

In some examples, the cover body is configured to maintain a substantially smooth surface during movement at the joint.

In some examples, the cover is configured to allow movement at the articulated joint over its full range of motion without significantly loading an actuator that drives movement at the articulated joint.

In some examples, the cover is configured to not back drive an actuator that drives movement at the articulated joint when stretched.

In some examples, the cover body includes a knitted material.

In some examples, the structural brace comprises a hand coupling configured to couple the cover to the automated hand.

In some examples, the hand coupling is made of moulded plastic.

In some examples, the hand coupling at least partly encircles an edge of the cover.

In some examples, the hand coupling is configured to couple to the palm of the automated hand.

In some examples, the structural brace comprises a wrist coupling configured to couple to a wrist that the automated hand is coupled to.

In some examples, the structural brace comprises a thumb coupling configured to couple to a thumb of the automated hand.

In some examples, the hand coupling is configured to be sandwiched between shell pieces of the automated hand.

In some examples, the wrist coupling is configured to be retained in a groove that encircles the wrist.

In some examples, the structural brace comprises one or more inward brace pieces located inward from the edges of the cover body to help maintain the shape of the cover.

In some examples, the inward braces comprise one or more hoops that fully or mostly encircle a part of the cover.

In some examples, the inward braces are made of moulded plastic.

In some examples, the wrist coupling and/or the thumb coupling are made of moulded plastic.

In some examples, the cover body is configured to allow water to pass through it such that water can drain from a region between the automated hand and the cover.

In some examples, the cover body includes a woven material.

In some examples, the cover is configured to approximate the shape of the part of a natural human hand that corresponds to the part of the automated hand that is covered by the cover.

In some examples, the structural brace is configured to maintain the shape of the cover when the cover is not on the automated hand.

In some examples, the cover body is substantially free of corrugations in use.

In some examples, the cover further comprises one or more reinforced regions configured to lie over protruding features or user input features of the automated hand.

In some examples, the cover further comprises one or more visual indicators configured to lie over user input features of the automated hand.

In some examples, the cover further comprises one or more seams, wherein the seams are sewn, knitted, glued, or joined using bonding tape.

According to another example there is provided an automated hand comprising:
a. a palm;
b. a thumb mounted to the palm of the hand, wherein the thumb is pivotally mounted to the palm at a first connection; and
c. an auxiliary support from an intermediate location along the thumb to a second connection on the palm.

In some examples, the second connection on the palm is spaced apart from the first connection.

In some examples, the first connection is at the base of the palm.

In some examples, the second connection is intermediate the base of the palm and the distal end of the palm.

In some examples, the intermediate location is at least 10% of the length of the thumb from an end of the thumb.

In some examples, the intermediate location is at least 25% of the length of the thumb from the end of the thumb.

In some examples, the thumb comprises two segments connected by an articulated joint.

In some examples, the intermediate location is near the articulated joint.

In some examples, the intermediate location is at the articulated joint.

In some examples, the intermediate location is distal of the articulated joint.

In some examples, the auxiliary support is flexible.

In some examples, the auxiliary support is a support arm.

In some examples, the support arm comprises a resilient material, for example a polymer.

In some examples, the second connection is a pivotal connection.

In some examples, the support arm is configured to connect to both sides of the thumb at the intermediate location.

In some examples, the support arm has a curved shape which can straighten under tension.

In some examples, a side of the support arm facing away from the base of the palm has a concave portion to assist gripping of objects.

In some examples, the support arm is configured such that it does not interfere with the palm or the thumb other than at the first and second connections as the thumb moves through its full range of motion.

In some examples, the auxiliary support is a cord.

In some examples, the thumb has a compliant portion between the first connection and the intermediate location.

In some examples, the first connection comprises a substantially rigid mount in or on the palm.

In some examples, the auxiliary support is frangible and wherein the thumb is connected to the palm via a safety pivot that allows the thumb to pivot freely in the extension direction upon breaking of the auxiliary support.

In some examples, the automated hand further comprises a catch configured to restrict pivoting of the thumb at the first connection when the thumb is under load.

According to another example there is provided an automated hand comprising:
a. a palm having a pivotal mount;
b. a digit pivotally mounted to the pivotal mount to pivot about a first axis, the digit being compliant at a compliance location distal of the pivotal mount; and
c. a catch configured to selectively restrict pivoting of the digit about the first axis when the digit complies at the compliance location under load.

In some examples, the digit is compliant about a second axis corresponding to a flexion-extension axis of the digit.

In some examples, the digit is a thumb.

In some examples, the first axis corresponds to an antepositioning-retropositioning axis of the thumb.

In some examples, the pivotal mount is substantially non-compliant about the second axis.

In some examples, the digit is disposed at or near a first side of the palm and wherein the catch is located between the digit and the first side of the palm.

In some examples, the catch includes a tooth attached to the digit and one or more recesses on the palm, wherein the tooth is driven towards the recess(es) when the digit complies at the compliance location.

In some examples, the tooth is biased away from the recess(es) when the digit is not under load.

In some examples, the load is a load in the extension direction of the digit.

In some examples, the digit comprises a compliant articulated joint at the compliance location.

In some examples, the catch is configured to restrict pivoting of the digit about the first axis when an external force is applied to the digit towards extension of the digit at the compliant articulated joint.

In some examples, the catch is configured to restrict pivoting of the digit about the first axis when a digit actuator drives the digit towards flexion of the digit at the compliant articulated joint.

In some examples, the catch includes a link arm that is coupled to a compliantly mounted gear, the compliantly mounted gear being mounted in a segment of the digit that is on the proximal side of the articulated joint and engaged with a gear on the distal side of the articulated joint.

In some examples, the gear on the distal side of the articulated joint is a drive gear that drives rotation of the digit at the articulated joint.

In some examples, the automated hand further comprises an actuator in the digit on the distal side of the articulated joint, the actuator being configured to drive the drive gear.

In some examples, the tooth is carried on the link arm.

In some examples, the bias is provided by a spring kinematically coupled to the link arm.

In some examples, the proximal portion of the digit comprises a compliant housing at the compliance location or another such compliance location.

According to another example there is provided an assembly comprising a wrist and an automated hand, the assembly further comprising:
a. a coupling tongue extending from the wrist or the automated hand, the coupling tongue having a distal portion and a proximal portion, the distal portion being wider than the proximal portion; and
b. a coupling clamp extending from the other one of the wrist and the automated hand, the coupling clamp configured to receive and clamp onto the coupling tongue to releasably couple the wrist to the automated hand.

In some examples, the coupling clamp comprises a clamp plate configured to be tightened onto the coupling tongue.

In some examples, the coupling clamp comprises one or more screw fasteners for tightening the clamp plate.

In some examples, clamping the coupling clamp onto the coupling tongue pulls the wrist and hand together.

In some examples, the assembly is configured such that the contact interface at which the coupling clamp and the coupling tongue contact each other when the coupling tongue is received in the coupling clamp is at an oblique angle to a longitudinal axis running through the wrist and the automated hand.

In some examples, wherein the coupling clamp and the coupling tongue have complementary faces configured to abut each other and restrict lateral movement of the hand with respect to the wrist.

In some examples, the coupling tongue is located within a pocket and wherein the coupling clamp is configured to fit snugly within the pocket such that the complementary faces include inner faces of the pocket and outer faces of the coupling clamp.

In some examples, the coupling tongue includes one or more ribs and wherein the coupling clamp includes one or more slots configured to fit snugly over the ribs such that the complementary faces include sides of the rib(s) and sides of the slot(s).

According to another example there is provided an automated hand assembly comprising:
a. an automated hand;
b. a wrist;
c. a releasable mechanical coupling for releasably mechanically coupling the wrist to the automated hand; and
d. a releasable electrical coupling for releasably electrically coupling the wrist to the automated hand;
wherein the releasable electrical coupling comprises a plurality of biased terminals on the automated hand or the wrist, each biased terminal being biased towards a complementary terminal on the other one of the automated hand and the wrist, each biased terminal being configured to electrically connect to the complementary terminal when the wrist is mechanically coupled to the automated hand.

In some examples, the biased terminals are spring loaded.

In some examples, the complementary terminals are pads.

In some examples, the pads are concave.

In some examples, the wrist comprises a cable, with the biased terminals or the complementary terminals being located at the end of the cable.

In some examples, the automated hand assembly further comprises a friction fit feature on the cable that configured to couple to a friction fit feature on the hand.

In some examples, the automated hand assembly further comprises a brace on the wrist, the brace configured to prevent electrical decoupling of the terminals when the cable and the hand are mechanically coupled.

In some examples, the wrist has a port formed in it to allow the cable to pass from one side of the wrist to the other.

In some examples, the automated hand assembly further comprises a rotary coupling for coupling the wrist to an arm socket, the rotary coupling configured to allow the wrist to rotate about a longitudinal axis of the arm socket.

According to another example there is provided a wrist for use with an automated hand, the wrist comprising:
a. a first coupling for coupling the wrist to an arm socket;
b. a second coupling for coupling the wrist to the automated hand;
c. a rotatable wrist joint between the first coupling and the second coupling, the wrist joint configured to rotate to allow the automated hand to flex or extend; and
d. a locking mechanism comprising a base and a locking plate configured to rotate relative to each other upon the rotation of the wrist joint, wherein the base has one or more recesses and the locking plate is selectively configurable between two states, a first state in which the locking plate is biased towards engagement with one of the one or more recesses of the base and a second state in which the locking plate is retained out of engagement with the recesses of the base; wherein the locking mechanism is configured to restrict the rotation of the wrist joint when the locking plate is engaged with one of the one of more recesses of the base.

In some examples, the locking mechanism further includes a locking button configured to be pushed along a first axis to select the configuration of the locking plate, the first axis being transverse to the direction in which the locking plate moves to enter the recess(es).

In some examples, the first axis lies along the rotational axis of the rotatable wrist joint.

In some examples, the locking plate has a recess for receiving a pin coupled to a locking actuator, the recess having a sloped surface, the plate being moved away from engagement with the recess when the pin rides over the sloped surface.

In some examples, the wrist further comprises a spring configured to bias the plate towards engagement with the recess(es).

In some examples, the one or more recesses are a plurality of recesses arranged radially around the rotational axis of the rotatable wrist joint.

In some examples, the locking plate is configured to move directly towards the rotational axis of the rotatable wrist joint to engage with the recesses.

According to another example there is provided a wrist joint for use with an automated hand, the wrist joint comprising:
a. a rigid sleeve with an aperture formed therein, the aperture being non-circular in cross section;
b. a rigid axle extending through the aperture, the axle being non-circular in cross-section; and
c. one or more resiliently deformable pieces in the aperture between the rigid sleeve and the rigid axle;
wherein the rigid axle has a neutral orientation with respect to the rigid sleeve, and wherein the one or more resiliently deformable pieces return the rigid axle towards the neutral orientation after rotation away from the neutral orientation.

In some examples, the rigid axle has a non-planar face adjacent to at least one of the resiliently deformable pieces.

According to another example there is provided a wrist joint for use with an automated hand, the wrist joint comprising:
a. a rigid sleeve with an aperture formed therein, the aperture being non-circular in cross section;
b. a rigid axle extending through the aperture, the axle being non-circular in cross-section; and
c. one or more resiliently deformable pieces in the aperture between the rigid sleeve and the rigid axle;
wherein the rigid axle has a non-planar face adjacent at least one of the one or more resiliently deformable pieces, the non-planar face being shaped to provide a neutral orientation of the rigid axle with respect to the rigid sleeve, and wherein the one or more resiliently deformable pieces return the rigid axle towards the neutral orientation after rotation away from the preferred neutral orientation.

In some examples, the non-planar face is concave.

In some examples, the non-planar face is configured to provide, for small deviations from the neutral orientation, a higher return torque towards the neutral orientation than a planar face would.

In some examples, the aperture in the rigid sleeve has a plurality of corners with a resiliently deformable piece in each corner.

In some examples, in cross section the perimeter of the aperture includes one or more part-circular arcs and one or more recesses; and each of the one or more recesses is configured to receive one of the one or more resiliently deformable pieces.

In some examples, in cross section the perimeter of the rigid axle includes one or more part-circular arcs and one or more recesses formed in it, wherein:
each recess of the rigid axle is configured to receive one of the one or more resiliently deformable pieces; and each of the one or more resiliently deformable pieces is held between one of the recesses of the aperture and one of the recesses of the rigid axle.

In some examples, the resiliently deformable pieces are elastomers or suitable polymers.

It is acknowledged that the terms “comprise”, “comprises” and “comprising” may, under varying jurisdictions, be attributed with either an exclusive or an inclusive meaning. For the purpose of this specification, and unless otherwise noted, these terms are intended to have an inclusive meaning—i.e., they will be taken to mean an inclusion of the listed components which the use directly references, and possibly also of other non-specified components or elements.

Reference to any document in this specification does not constitute an admission that it is prior art, validly combinable with other documents or that it forms part of the common general knowledge.

### BRIEF DESCRIPTION OF THE DRAWINGS

The accompanying drawings, which are incorporated in and constitute part of the specification, illustrate embodiments of the invention and together with the general description of the invention given above and the detailed description of embodiments given below serve to explain the principles of the invention.

FIG. 1 is a perspective view of an automated hand according to one example;

FIG. 2 is another perspective view of the automated hand of FIG. 1;

FIG. 3 is a partly exploded view of the automated hand of FIG. 1;

FIG. 4 is a perspective view of part of an automated hand of FIG. 1;

FIG. 5 is a perspective view of a connector mounting arrangement according to one example;

FIG. 6 is another perspective view of the connector mounting arrangement of FIG. 5;

FIG. 7 is a cross sectional view through the connector mounting arrangement of FIG. 6;

FIG. 8 is a perspective view of a connector mount according to one example;

FIG. 9 is a partly exploded view of the connector mount of FIG. 8;

FIG. 10 is a front view of part of a connector mount according to one example;

FIG. 11 is a top view of an actuator assembly according to one example;

FIG. 12 is a cross sectional view through the actuator assembly of FIG. 11;

FIG. 13 is a partly exploded view of a gear bearing arrangement according to one example;

FIG. 14 is a partly exploded view of a digit drive arrangement according to one example;

FIG. 15 is a partly exploded view of a digit according to one example;

FIG. 16 is a top view of a digit according to one example;

FIG. 17 is a cross-sectional view through the digit of FIG. 16;

FIG. 18 is a perspective view of a cover according to one example;

FIG. 19 is another perspective view of the cover of FIG. 18;

FIG. 20 is a side view of a wrist assembly connected to a palm chassis according to one example;

FIG. 21 is a side view of the wrist assembly and palm chassis of FIG. 20 separated from each other;

FIG. 22 is another side view of the wrist assembly and palm of FIG. 20 separated from each other;

FIG. 23 is an end view of the palm chassis of FIG. 20;

FIG. 24 is a partly exploded view of the wrist assembly of FIG. 20;

FIG. 25 is a perspective view of a wrist according to one example;

FIG. 26 is a view of two components of the wrist of FIG. 25;

FIG. 27 is a top view of the wrist of FIG. 25;

FIG. 28 is a cross-sectional view of the wrist of FIG. 20;

FIG. 29 is a partly exploded view of the wrist of FIG. 20;

FIG. 30 is a perspective view of part of a locking mechanism according to one example;

FIG. 31 is a perspective view of a wrist assembly connected to a palm chassis according to another example;

FIG. 32 is a perspective view of a thumb assembly according to one example;

FIG. 33 is another perspective view of the thumb assembly of FIG. 32;

FIG. 34 is a top view of the thumb assembly of FIG. 32;

FIG. 35 is a perspective view of components of a locking assembly according to one example;

FIG. 36 is an exploded view of the thumb assembly of FIG. 32;

FIG. 37 is another exploded view of the thumb assembly of FIG. 32;

FIG. 38 is another view of the thumb assembly of FIG. 32;

FIG. 39 is a cross sectional view of an alternative wrist to that of FIG. 20;

FIG. 40 is a perspective view of components of a locking mechanism according to another example; and

FIG. 41 is a perspective view of components of a thumb assembly according to another example.

### DETAILED DESCRIPTION

The present specification describes and claims several aspects of automated hands and components for use with automated hands. Any combination of the described and claimed aspects and components may be provided together in an automated hand or in an assembly that includes an automated hand, except when it is clear from the context that the aspects or components are exclusively to be used as alternatives to each other.

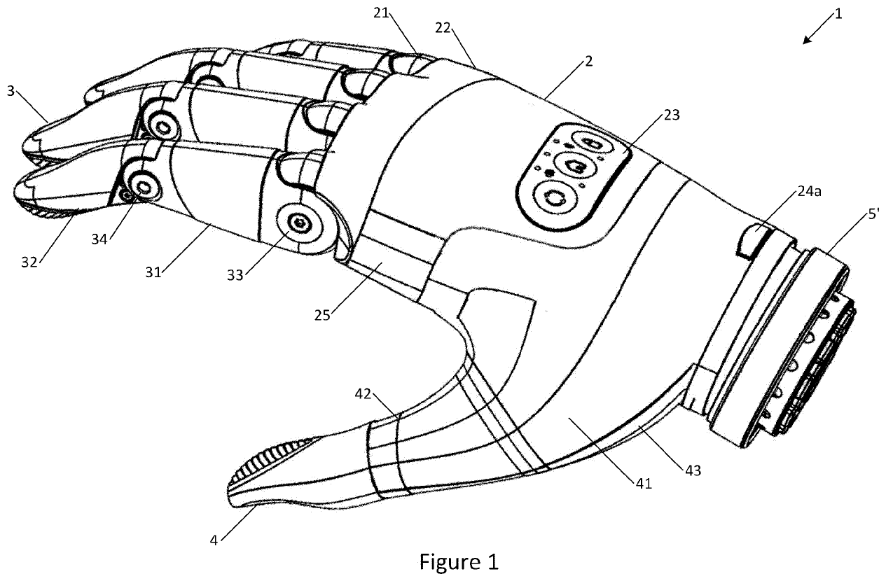

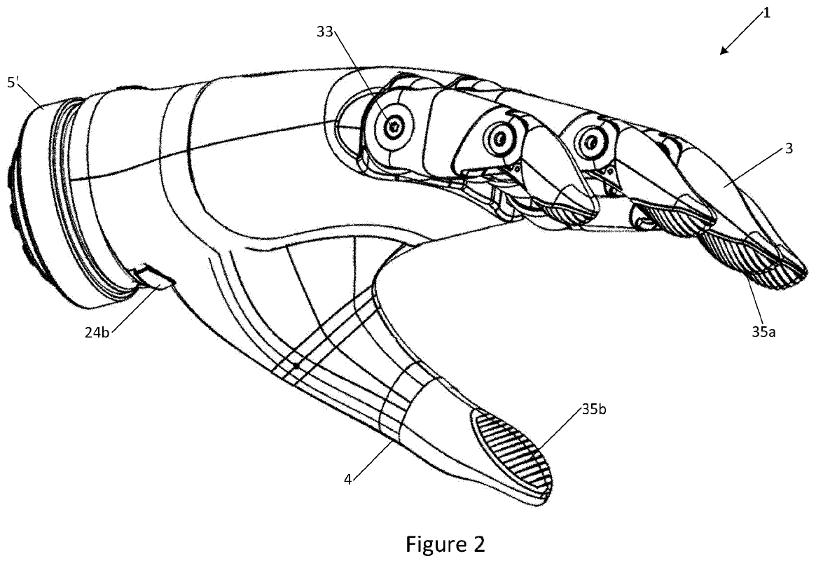

FIGS. 1 and 2 illustrate an automated hand 1 according to an example embodiment.

Size can be an important consideration in the design of automated hands. Overly large automated hands may be heavy, inconvenient and ill-matched to the user.

The automated hand 1 can have several space-saving features that may allow the automated hand 1 to be made relatively small and compact.

The automated hand 1 has a palm 2. Attached to the palm 2 are digits 3, 4. The palm 2 and digits 3, 4 can be arranged to correspond to the palm and digits of a natural human hand.

In this example, the digits include four fingers 3 and one thumb 4. The fingers 3 of this example are arranged like the four fingers of a natural human hand. The fingers 3 of this example may differ from each other like the fingers of a natural human hand. In particular, the fingers 3 could have different sizes like the fingers of a natural human hand.

The thumb 4 of this example is arranged like the thumb of a natural human hand. The thumb 4 can be movable to oppose the fingers 3 in a similar way to the thumb of a natural human hand.

Overall, the automated hand 1 of this example is arranged to be generally anatomically correct. In other examples, the automated hand 1 may be less anatomically correct and arranged differently from a natural human hand. For example, the automated hand 1 may have more or fewer than four fingers and more or fewer than one thumb.

The fingers 3 of this example are each made up of two sections 31 and 32. Section 31 is referred to herein as the proximal phalanx 31. Section 32 is referred to herein as the distal phalanx 32. The proximal phalanx 31 and distal phalanx 32 are connected by an articulated joint 34, serving as a knuckle. The fingers 3 in this example differ from the fingers of a natural human hand in that a natural human hand has three phalanges—the proximal, intermediate and distal phalanges. In some examples, each finger 3 could have more or fewer than two phalanges, for example it could have proximal, intermediate and distal phalanges like a natural human hand.

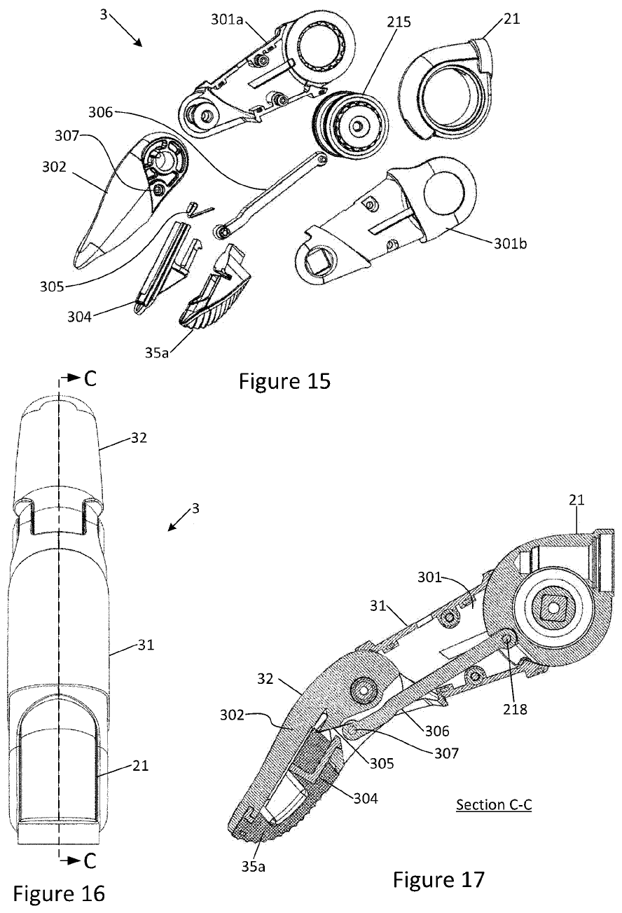

As best shown in FIG. 2, a pad 35a can be provided at the end of each finger 3. This may improve grip. The pads 35a may also help enable the fingers 3 to operate a touchscreen as will be detailed further with reference to FIGS. 15-17.

The fingers 3 are connected to the palm 2 by respective articulated joints 33. The articulated joints 33 can be provided in respective knuckles 21. As will be detailed further with reference to FIGS. 4-10, the fingers 3 can be compliantly mounted to the palm 2.

The thumb 4 of this example is made up of two sections 42 and 43. Section 43 is referred to herein as the thumb metacarpal. Section 42 is referred to herein as the thumb phalanx. The sections 42 and 43 can be connected to each other by an articulated joint (not shown in FIGS. 1 and 2). The thumb 4 can be connected to the palm 2 by an articulated joint (not shown in FIGS. 1 and 2). The thumb 4 of this example differs from the thumb of a natural human hand in that a natural human thumb has a metacarpal and proximal and distal phalanges with articulated joints between them. In other examples, the thumb 4 could have more or fewer sections. For example, it could have a metacarpal, a proximal phalanx and a distal phalanx like a natural human thumb.

As best shown in FIG. 2, a pad 35b can be provided at the end of the thumb 4. This may improve grip. The pad 35b may also enable the thumb 4 to operate a touchscreen as will be discussed with reference to FIGS. 15-17.

In FIGS. 1 and 2, the thumb 4 is shown covered by a cover 41. The cover 41 can cover the articulated joint connecting the thumb metacarpal 43 to the palm 2 and/or the articulated joint connecting the thumb metacarpal 43 to the thumb phalanx 42.

Also shown in FIGS. 1 and 2 is a wrist 5′ for connecting the automated hand 1 to a user's arm. The automated hand 1 may be suitable for use with a range of different wrists. In the example of FIGS. 1 and 2, the wrist 5′ is a quick-disconnect wrist. The wrist 5′ of this example can be operated to disconnect the automated hand from an arm coupling by simultaneously pressing at 24a and 24b. Patches or other visual indicators may be provided on the cover 41 in the regions 24a and 24b to indicate where to press to disconnect the wrist 5′.

The palm 2 in this example is partly covered by a faceplate 25. In the faceplate 25 is user interface panel 23. The user interface panel 23 may have input devices such as buttons. The user interface panel 23 may have output devices such as lights or a display screen. The user interface panel 23 may have a touchscreen serving as an input/output device.

A fascia 22 can also be provided on the palm 2 near the knuckles 21. This may be moulded such that it fits snugly onto the palm 2 and over the knuckles 21.

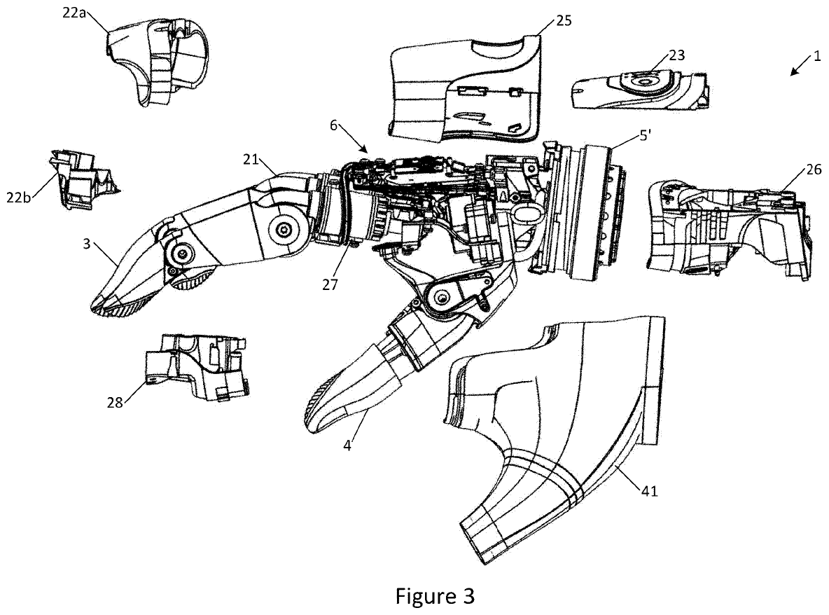

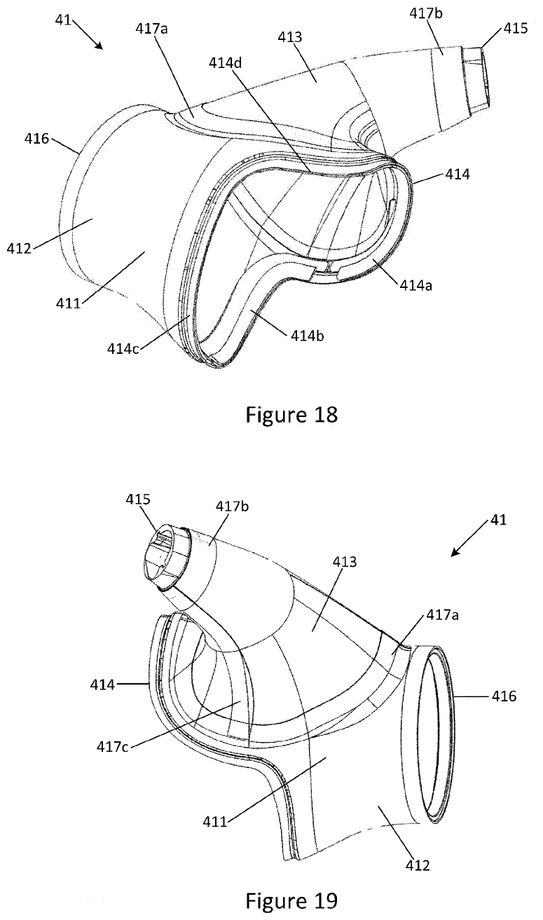

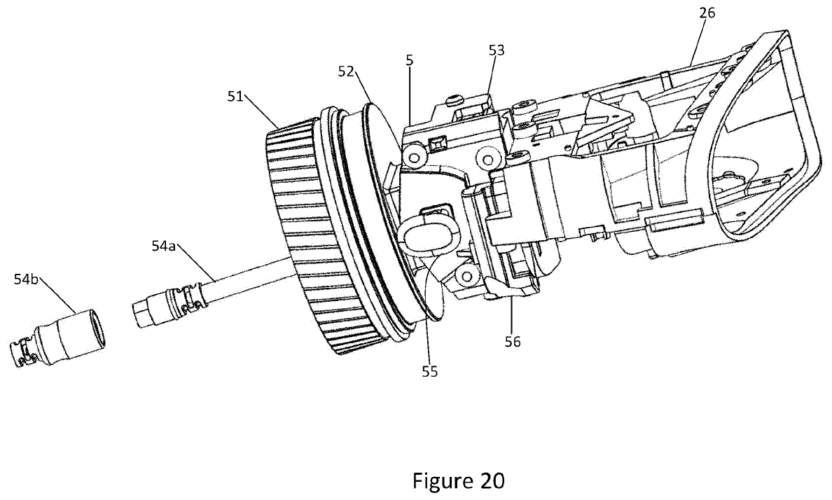

In FIG. 3, the automated hand 1 is shown with the fascia 22 (made up of upper part 22a and lower part 22b), faceplate 25, interface panel 23 and cover 41 separated from the rest of the hand 1. With these parts separated, the interior 6 of the palm 2 is visible. Control electronics and digit actuators can be sealed within the interior 6. Also shown separated from the rest of the hand 1 are the palm chassis 26 and palm cover 28, which surround the interior 6 of the palm 2 when assembled. The faceplate 25, interface panel 23, fascia 22 and cover 41 can be assembled onto the palm over the chassis 26 and palm cover 28. The cover 41 can be secured to the palm as described in more detail with reference to FIGS. 18 and 19. For example, the cover 41 may be clipped beneath the faceplate 25. The wrist 5′ can be secured to the palm chassis 26 as will be described in more detail with respect to FIGS. 20-22 and 31.

FIG. 3 shows a connector mount 27, which is located beneath the fascia 22 in the automated hand 1 when assembled. The connector mount 27 mounts one or more connectors to which one or more digits are connected. In the example of FIG. 3, the connector mount 27 mounts the four fingers 3 as shown in FIG. 4.

### Digit Mounting Arrangement

In automated hands, it can be desirable to seal interior portions against water intrusion. It can also be desirable to compliantly mount parts of the hands, such as digits. Digits may be knocked, pulled and pushed in use. Providing compliance to the digit mounts allows them to move somewhat in these scenarios without incurring damage at their point of mounting and without damaging parts of the hand to which they are mounted. This may also allow them to passively conform to the shape of a gripped object and feel more natural, for example in a handshake. However, it may also be desirable to ensure that digits are accurately located and oriented with respect to each other and the rest of the hand, which may be challenging when the digits are compliantly mounted and may require significant “tuning” of the digit control algorithms and/or adjustment of the digit positions after mounting to ensure that they accurately execute desired grips. Additionally, compliant mounting arrangements may in some cases also provide a sealing function and it may be challenging to design compliant elements that provide both good sealing and good compliance. It may also be desirable to mechanically control the limits and other parameters of the compliant movement of the digits more precisely than in other automated hands.

FIG. 4 shows the four fingers 3 of the exemplary automated hand 1 of FIGS. 1-3 mounted using the connector mount 27. In other examples, more or fewer fingers and/or one or more thumbs may be mounted using the connector mount 27.

As shown in FIG. 4, connectors 29 are mounted to the mount 27. The fingers 3 are connected to respective connectors 29 at the knuckles 21. The fingers 3 are thereby mounted to the palm via the connectors 29 and mount 27. In some examples, the connectors 29 can form part of a digit drive that drives the digit(s) to flex and/or extend. For example, each connector 29 could include an actuator. In other examples, each connector 29 could include one or more cables or one or more link arms of a linkage. In these examples, the cable or link arm may be located between the digit and an actuator that is placed further back in the palm, in the wrist, or behind the wrist. In other examples, the digit may include the actuator (e.g. motor) that causes the movement of the digit with respect to the palm. In this example, the respective connector may be “passive” and not actuate movement of the finger. For example, the connector may include a mounting arm with a fixed gear at its end, with the actuator in the digit having a driven gear that engages with the fixed gear and rotates to drive movement of the digit.

Each connector 29 is mounted such that it can move within the mount 27 when a force is applied to the digit that extends from that connector 29. The mount 27 may allow for rotation of the connector 29 in the plane of the palm (i.e. about an axis that is transverse or approximately normal to the plane of the palm), in a plane normal to the plane of the palm (i.e. about an axis that runs approximately laterally through the palm), and/or about the longitudinal axis of the connector 29. The mount 27 may allow for translation of the connector 29 in the plane of the palm or normal to the plane of the palm. Each connector 29 may thereby be mounted with three degrees of translational freedom and three degrees of rotational freedom, although in some examples the connector 29 may be mounted with fewer degrees of rotational or translational freedom.

The mount 27 can be located at or near the front of the palm, as shown in FIG. 3. The mount 27 can be generally in the part of the palm that corresponds to the metacarpal region of a natural human hand. The connectors 29 can correspond to metacarpals of a natural human hand. In a natural human hand, metacarpal bones extend through the palm towards the knuckles, at which they connect to digits. The connectors 29 may similarly extend through the palm 2 of the automated hand 1 to connect to the digits 3, 4 of the automated hand 1. In the detailed example described herein, the mount 27 mounts the four fingers 3 of the automated hand via connectors 29 that correspond to the four metacarpals of a natural human hand that connect to the four fingers. In other examples, the mount could mount more or fewer fingers 3 or it could mount the thumb 4 alone or the thumb 4 in combination with one or more fingers 3.

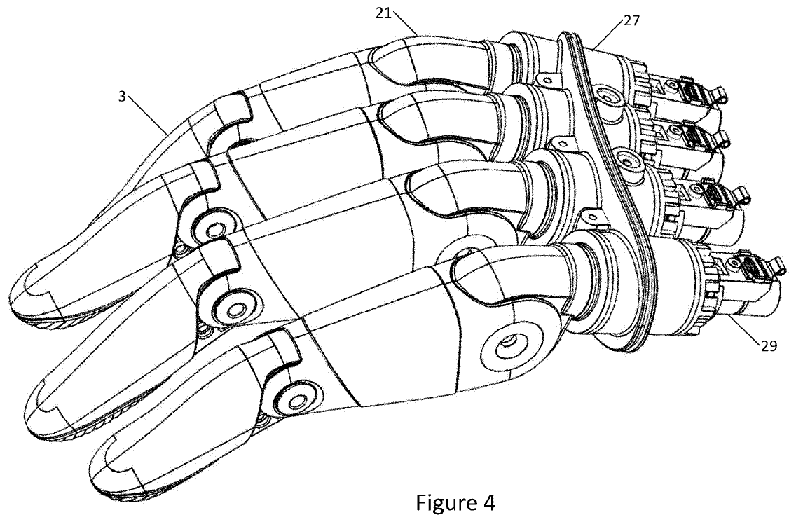

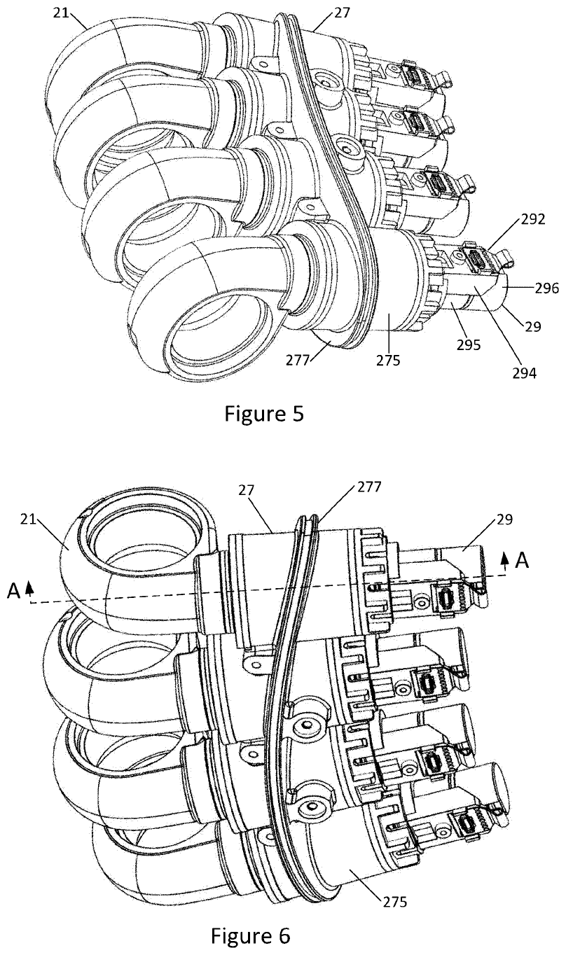

FIGS. 5 and 6 show the mounting arrangement for the digits in more detail. The connectors 29 in this example include actuators in the form of motors 295. The connectors 29 in this example drive flexion and extension of the digits. Various types of motors may be suitable. In one example, the motors 295 are brushless DC (BLDC) motors. Each motor 295 is provided with an encoder 296 and a printed circuit board (PCB) 292 on a PCB mount 294. The connectors 29 extend through the mount 27 and terminate in the knuckles 21 to which the digits are mounted. The mount 27 can include a rigid mount for each connector 29. In the example shown, the connector mounts are integral with each other, forming a single rigid mount 275 for all of the connectors 29. In other examples, separate rigid mounts for the different connectors 29 may be used. The rigid mount 275 has a flange 277 around it. The flange 277 is dimensioned to fit within the palm chassis 26 (shown in FIG. 3) when the hand is assembled. The flange 277 has a groove formed in its peripheral edge for receiving an O-ring (not shown) that seals to the palm chassis 26 when assembled. This may prevent water or other liquids from entering the interior of the palm via the front of the palm. Other seals such as gaskets, elastomer blocks or silicone sealant or the like could be used in place of or in addition to an O-ring. In other examples, a seal such as an O-ring may be provided on the palm chassis to seal against the rigid mount(s) 275, or one or more intermediate members could be placed between the chassis 26 and the rigid mount(s) 275 with seals between the intermediate member(s), the palm chassis 26 and the rigid mount(s) 275.

In the example shown the rigid mount 275 is a separate member from other parts of the palm to which it is attached. In other examples, one or more rigid mounts may be formed integrally with or as part of other structural member(s) of the palm. For example, a modified palm chassis may incorporate the rigid mount. In such an arrangement, the palm chassis may be provided in two parts that are fastenable together to define an aperture for each connector. In another example, one or more palm housing/shell pieces could have outer surfaces that provide the exterior body of the palm and rigid inner surfaces structured to provide the rigid mounts.

The rigid mount or mounts 275 can be made of any suitable material that can provide sufficient rigidity. In some examples, the rigid mount or mounts 275 are made of metal. In some examples, the metal is aluminium.

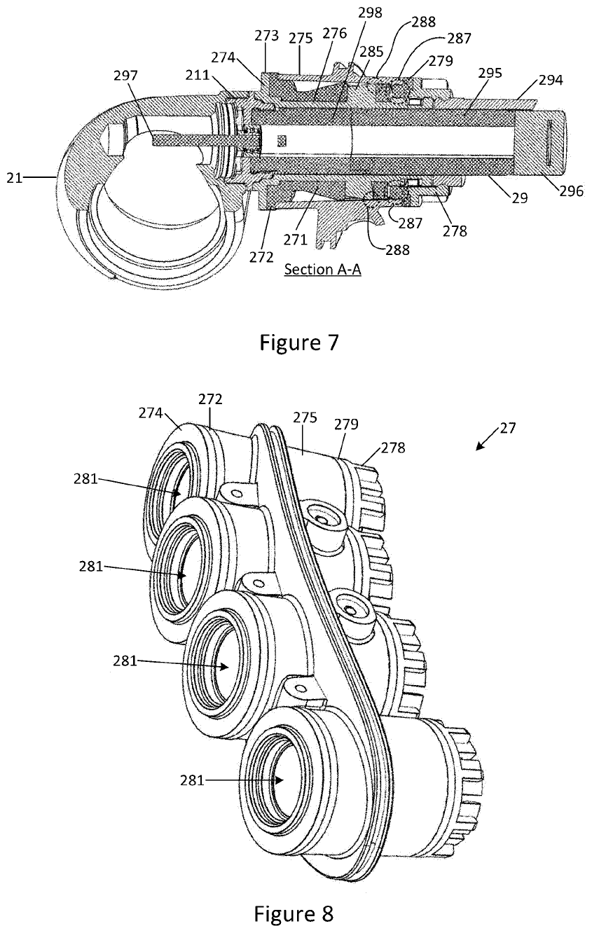

FIG. 7 is a cross-sectional view through the mounting arrangement of FIG. 6, taken along line A-A. This shows the detailed construction of one example of the mounting arrangement for a single connector 29.

The rigid mount 275 has within it a sleeve 271. The sleeve 271 can be resiliently deformable such that it can deform by an appreciable amount under forces having magnitudes that would be typically encountered in normal use of the automated hand. These forces could be experienced when a digit attached to the connector 29 is knocked, pushed, pulled, twisted etc. or otherwise experiences shocks or stresses. The resilience of the sleeve 271 can also return it towards its neutral or undeformed state after removal of the forces, although it will be appreciated that in some cases there may be some hysteresis or plasticity associated with the sleeve 271 which may result in it not returning perfectly to the neutral or undeformed state.

Suitable materials for the resiliently deformable sleeve 271 include elastomers, rubbers, silicone, or polymers; polyurethane, hydrocarbon-, fluorocarbon- or silica-based elastomers; thermoset elastomers; thermoplastic materials such as thermoplastic elastomers; and thermoset rubbers; foamed compositions of one or more of these materials; and alloys or blends of two or more of these materials. The sleeve material can be selected to have a Dynamic Mechanical Thermal Analysis (DMTA) damping factor between about 0.05 and about 0.8 over a temperature range of about −20° C. to about 100° C.; or between about 0.05 and about 0.5 over a temperature range of about −20° C. to about 100° C. The sleeve material can be selected to have a resilience of between about 20% and about 60%, which may be measured according to the ASTM D2632 standard. The sleeve material can be selected to have a Shore A hardness of between about 10 and about 90; a Shore A hardness of between about 30 and about 60; a Shore A hardness of about 30; or a Shore D hardness of between about 40 and about 90.

The resiliently deformable sleeves 271 has an aperture for receiving a respective connector 29. The apertures are generally indicated by the arrows 281 in FIG. 8. Providing the resiliently deformable sleeve 271 between the connector 29 and the rigid mount 275 allows the connector 29 to move—for example rotate and translate-within the rigid mount 275 while the rigid mount remains substantially unmoved. Because the rigid mount 275 is substantially unmoved, the seal formed between the rigid mount 275 and the palm is not compromised when the connectors 29 move about within the mount 275.

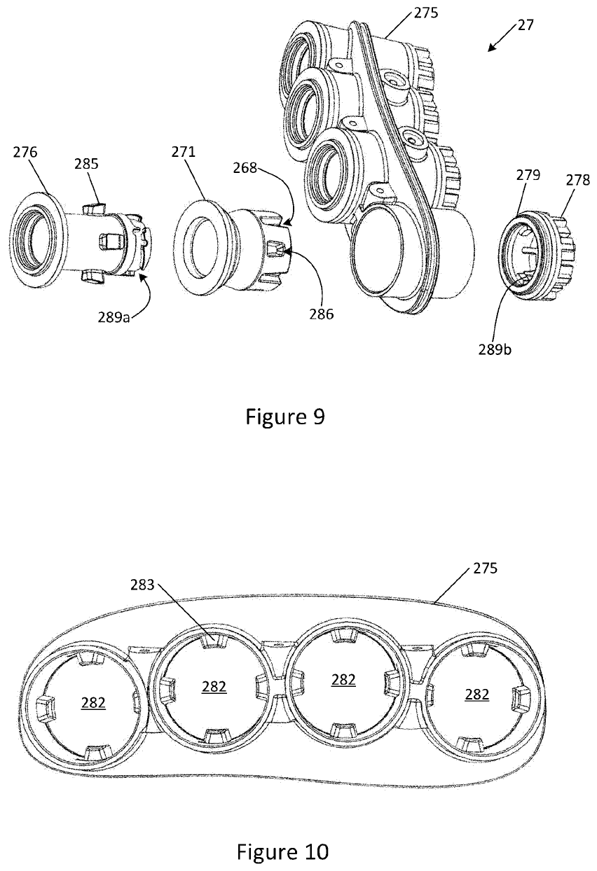

In the example of FIG. 7, a rigid sleeve 276 is placed between the connector 29 and the resiliently deformable sleeve 271. The rigid sleeve 276 can have features formed on it to assist with mounting of the connector 29 and controlling of the movement of the connector 29 in response to forces. The rigid sleeve 276 of this example includes projections 285 that project outwards from the rigid sleeve 276 towards the rigid mount 275. The resiliently deformable sleeve 271 can have corresponding apertures (labelled 286 in FIG. 9) for the projections 285 to pass through. The projections 285 provide a pivot 288 for rotations of the connector 29. The projections 285 and the inner surface of the rigid mount 275 are in close proximity with each other and form bearing surfaces when in contact with each under rotations at the pivot 288. There may be small gaps between the projection 285 and the rigid mount in the neutral position, which may allow some lateral translation of the connector 29 under applied forces. In other examples, one or more projections may project inwards from the inner surface of the rigid mount 275 towards the rigid sleeve to form the pivot 288. Various numbers of complementary bearing surfaces may be provided to act as pivots.

The rigid sleeve 276 can attach to the retainer rings 211 and 278 at the front and rear, respectively, of the connector 29. The connector 29 can also attach to the retainer ring 211, to which the knuckle 21 can attach. The rigid mount 275 can be held between the retainer ring 278 and a flange 274 at the front of the rigid sleeve 276. The rigid sleeve 276 can thus mount the connector 29 to the rigid mount 275 and can mount the knuckle 21 to the connector 29.

A resilient flange 272 of the resiliently deformable sleeve 271 is placed between the flange 274 of the rigid sleeve 276 and the front of the rigid mount 275. A sealing ring 279 is placed between the retainer ring 278 and the back of the rigid mount 275. The sealing ring 279 can provide the main seal that prevents water or other liquid from entering the interior of the hand via the digit mounts 27. In one example, the sealing ring 279 is overmoulded onto the retainer ring 278, although in other examples it may be provided as a separate element. The sealing ring 279 is configured to provide a seal 287 between the rigid mount 275 and the connector 29. In this example, the seal 287 is between the rigid mount 275 and the rigid sleeve 276. The seal 287 can be located near the pivot 288. Because the seal 287 is near the pivot 288, movement of the connector 29 (or rigid sleeve 276) with respect to the rigid mount 275 due to pivoting of the connector is low at the seal 287. The means that the sealing ring 279 does not need to accommodate large variations in the spacing between the sealed elements, thereby improving sealing reliability at this point. The sealing and compliance functions of the automated hand are separated, with the compliance being provided by the resiliently deformable sleeve 271 and the seal being provided by the sealing ring 279. This may allow the compliant mount features to be optimised for compliance without compromising reliability of the seal provided by the seal features and vice-versa.

Also shown in FIG. 7 is the PCB mount 294, which is connected to the connector 29 by the retainer ring 278 in this example. FIG. 7 also shows the motor 295 of the exemplary connector 29, as well as the motor encoder 296, motor transmission 298 and motor output shaft 297. Typically, the encoder 296, transmission 298 and output shaft 297 would be provided in a single unit with the motor 295.

FIG. 8 shows the mount 27 in isolation. The flange 274 of each rigid sleeve (labelled 276 in FIG. 7) is shown at the front of the mount 27, with the resilient flange 272 of the resiliently deformable sleeve (labelled 271 in FIG. 7) held between the flange 274 and the rigid mount 275. At the back of the mount 27, the sealing ring 279 is shown held between the retainer ring 278 and the rigid mount 275. The apertures through the resiliently deformable sleeves are indicated by arrows 281.

FIG. 9 shows the exemplary mount 27 for four connectors with the mounting arrangement for one connector exploded. The shows the rigid sleeve 276, resiliently deformable sleeve 271, sealing ring 279 and retainer ring 278 for one connector—in this example the connector for the index finger.

As shown in FIG. 9, there can be several projections 285 from the rigid sleeve 276 and a corresponding number of apertures 286 in the resiliently deformable sleeve. In this example, there are four projections 285 evenly spaced around the perimeter of the rigid sleeve 276, although one is obscured by the body of the rigid sleeve 276.

Twist lock features 289a and 289b are provided on the rigid sleeve 276 and retainer ring 278, respectively. In this example, the rigid sleeve 276 has a complex groove 289a into which fit tabs 289b on the inside of the retainer ring 278. In this example, the sealing ring 279 is moulded onto the retainer ring 278. To assemble the connector mounting arrangement, the rigid sleeve 276 is inserted into the resiliently deformable sleeve 271, which is itself inserted into the aperture (labelled 282 in FIG. 10) of the rigid mount 275. The retainer ring 278 is then twist locked to the rigid sleeve 276.

In other examples, the rigid sleeve 276 may be omitted and one or more of the features of the rigid sleeve 276 described above may instead be provided on the exterior of the connector itself.

FIG. 10 shows the rigid mount 275 in isolation. The rigid mount 275 has apertures 282 for receiving the connectors and resiliently deformable sleeves. Within the apertures 282 are projections 283 that project inwardly towards the connector. These projections slot into the indents 268 (shown in FIG. 9) in the resiliently deformable sleeves during assembly. These act as rotation restraints to limit rotation of the connector about its longitudinal axis, although some rotation is allowed by resilient deformation of the resiliently deformable sleeve.

The apertures 282 can be dimensioned to set limits on the maximum lateral rotation of the connector (i.e. in the plane of the palm and in a plane normal to the plane of the palm). The wider the aperture 282 is in a given direction, especially at its ends (away from the pivot), the greater the maximum rotation is. The maximum rotations in different directions can be set separately by setting the widths in different directions, i.e. by making the aperture non-circular in cross section. This allows the maximum connector rotation in one direction (e.g. corresponding to splaying of fingers) to be different from rotation in another direction (e.g. corresponding to rotation in the flexion-extension direction).

In an alternative example (not shown in the drawings), a barrier may be provided around a part of the connector that is not within the mount. For example, a rigid ring could be provided around the proximal portion of the connector (i.e. rearward of the mount) to limit the rotation of the connector at this point. This could be dimensioned to control the limits of rotation in each direction.

### Worm Bearing

Worm drives are commonly used in automated hands to drive digit movement. A worm can be placed on a drive motor in the palm or digit. The worm can engage with a worm wheel on the other of the palm and the digit. When using worm drives to rotate worm wheels, axial forces are generated on the worm. Typically, bearings are used on both sides of the worm to resist the axial forces. These bearings resist axial forces in one direction each. To resist the quite large axial forces on the worm, the bearings need to both be quite large. In automated hands, space can be extremely limited and having a large bearing at each end of the worm can hamper the design of compact hands. In particular, it may require a knuckle to be quite large to house the large bearing at the distal end of the worm. Additionally, having two different bearings resisting axial forces requires careful control of the tolerances to axial play of both bearings. It may be advantageous to provide a worm drive that does not require two separate bearings to resist axial forces in both directions along the longitudinal axis of the worm.

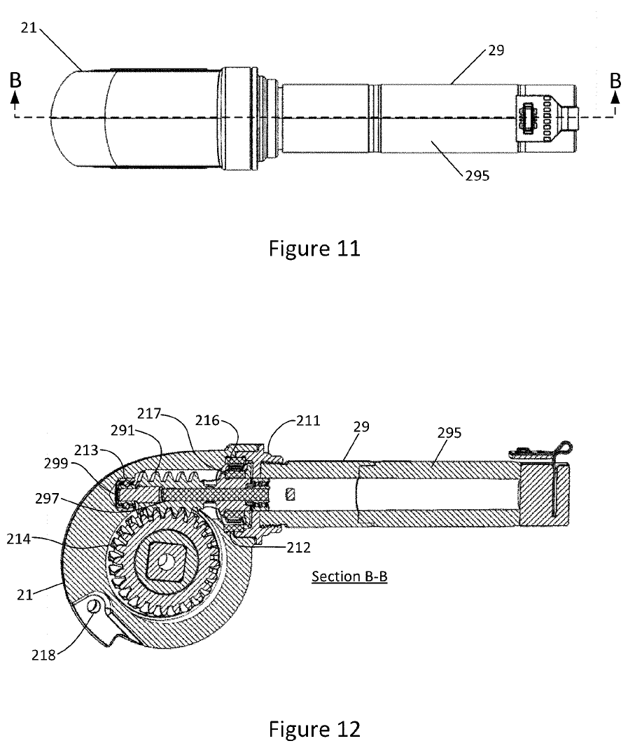

FIGS. 11 and 12 show an actuator assembly made up of the connector 29 and knuckle 21. The connector 29 is connected to the knuckle 21 by the retainer ring 211. In this example, the assembly is connected to a finger of the automated hand, although it could also be used with a thumb.

FIG. 12 is a cross-sectional view along line B-B of FIG. 11. Knuckle 21, in combination with retainer ring 211, serves as a gear housing for worm 291 and worm wheel 214. The worm 291 is supported by bearing 212. The bearing 212 is designed to be able to resist the axial forces on the worm 291 in both axial directions (i.e. left-to-right or right-to-left in the orientation shown in FIG. 12) when the worm 291 is rotated by the motor 295 of the connector 29. The bearing 212 can be selected to be sufficiently large to resist the forces expected to be encountered during operation of the automated hand without the need for a second bearing to assist in resisting the axial forces. Generally speaking, the larger a bearing is, the greater the axial loads it can resist. The bearing 212 can be a deep-groove ball bearing, which is particularly well suited to handling axial forces.

Removing the need for a second bearing resisting axial forces may reduce the size of the worm drive, allowing for a more compact hand design. In particular, the size of the gear housing (in this example provided by knuckle 21) may be reduced.

In the example of FIG. 12, the bearing 212 that resists axial forces is between the motor 295 and the worm 291. This allows the size of the worm drive at the other (distal) end of the worm 291 to be reduced. In this example, a second bearing 213 is used at the distal end of the worm 291. This bearing 213 can be small as it does not need to resist axial forces. In this example, the bearing 213 substantially only resists radial forces, which does not require the bearing 213 to be large. The second bearing 213 can be smaller than the bearing 212. The second bearing 213 can have a diameter that is about two thirds of the diameter of the bearing 212 or less, for example about half of the diameter of the bearing 212.

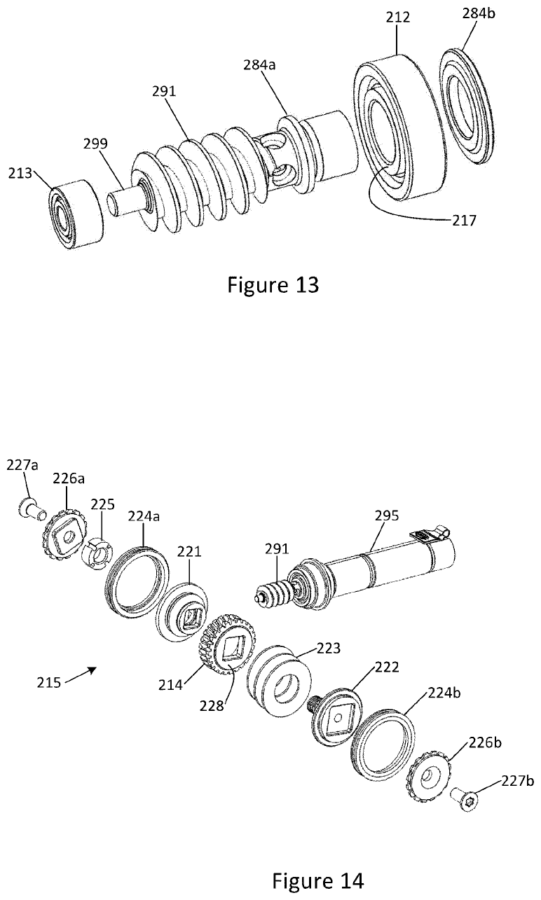

The worm 291 can be prevented from moving axially relative to the bearing 212. Axial forces on the worm 291 can be transferred to the bearing 212. In this example, flanges (shown as 284a and 284b in FIG. 13) are secured to or formed on the worm 291. The flanges 284a and 284b are located on each side of the bearing 212 to capture and retain the bearing 212 between the flanges on the worm. In some examples, one or both of the flanges 284a and 284b can be welded to the worm (e.g. by laser welding). In the example of FIG. 13, the flange 284a is integrally formed on the worm 291 and flange 284b is welded to the worm 291. In this example, the flanges 284a and 284b are secured to the worm 291 in close contact with the inner race 217 of the bearing. The outer race 216 of the bearing 212 can be held between the knuckle 21 and the retainer ring 211.

The second bearing 213 may be free to move axially with respect to the worm 291. In this example, the second bearing 213 is mounted on bearing shaft 299 but is free to slide axially along the shaft 299. The bearing shaft 299 can be fixed to the worm 291, for example by a press fit. This isolates the second bearing 213 from axial forces so that it provides substantially no resistance to them.

The motor 295 could be provided in either the palm or digit of an automated hand. In an exemplary automated hand, the motor 295 is in the palm. In this arrangement, the worm wheel 214 would be connected to a digit to drive rotation of the digit. The digit could have two sections, e.g. proximal and distal phalanges, that are connected to each other by an articulated joint. In the example of FIG. 12, the knuckle 21 includes a mount 218 for a link arm of a linkage that connects to the distal phalange of the digit. This allows rotation of the proximal phalanx at the knuckle to drive rotation of the distal phalanx at its articulated connection to the proximal phalanx.

FIG. 13 shows components of the worm drive separated from each other. These include the worm 291, bearings 212 and 213, flanges 284a and 284b and bearing shaft 299. The size difference between the larger bearing 212 and smaller bearing 213 can be seen here. When assembled, the bearing 213 is placed on the bearing shaft 299 and may be secured to it, with the shaft 299 being slidable within the worm 291. In an alternative arrangement, the bearing shaft 299 could be secured to or integrated with the worm 291 or the motor output shaft 297, with the bearing 213 being slidable on the bearing shaft 299.

FIG. 14 shows a drive for a digit that includes a worm drive and a digit drive assembly 215. The motor 295 drives worm 291, which in turn drives the worm wheel 214. The worm wheel 214 drives rotation of the output wheels 226a and 226b via a clutch assembly made up of threaded ring 225, drive shaft 222, clutch core hub 221, clutch core slider 228, and disc springs 223. The clutch assembly is fixed to the digit using screws 227a and 227b. Output bushes 224a and 224b are also provided at either side of the digit drive assembly 215. The clutch assembly can transfer rotation from the worm wheel 214 to the output wheels 226a, 226b while allowing slip between them when a torque between them is above a threshold. This may help to protect components of the actuator assembly, such as housings, bearings, gears and gear teeth, from high loads.

### Touchscreen Compatibility

Touchscreen devices are ubiquitous in the modern world, however these typically rely on electrical properties of a natural human body to detect a touch. Users of automated hands may be unable to operate these touchscreens if their automated hand does not have special arrangements to approximate the relevant electrical properties of a natural human hand or body. For example, capacitive touchscreens can detect a touch by the effect of a human body acting as a parasitic capacitance to ground. Automated hands may be unable to provide sufficient parasitic capacitance to ground to register a touch on these touchscreens.

Some prosthetics have sought to address the issue of operating capacitive touchscreens by providing a conductive connection between a fingertip of the automated hand and the user's body. This can be difficult to implement given the different types of stumps and partial hands that the prosthetics are to be fitted to. These designs may also require dedicated conductive wires and the like to be incorporated at the time of manufacture, making them unsuitable for retrofit applications. There may also be safety concerns relating to electro-static discharge with these designs. These designs are typically avoided in automated hands.

Some automated hands provide conductive substance such as paint, glue or the like at the tip of a finger. This can provide a conductive path from the tip to electrical components such as a motor in the hand, via a complex electrical path that may include dedicated wires electrically connecting the conductive substance to a terminal of the electrical component. These conductive substances can be relatively hard, meaning that they may only make contact with the screen over a very small contact area. This means that touches may not be reliably recognised, especially when the angle of the finger on the screen is non-optimal. They may also require an electrical connection between the conductive substance to a motor terminal in order for them to work.

Some automated hands provide a conductive coating or tip on a finger that does not electrically connect to other finger components. In these designs, the conductive coating or tip itself is intended to emulate the electrical properties of a natural human body. Without connection to any other conductive members of the hand, it may be difficult to provide sufficient parasitic capacitance to reliably register a touch on a capacitive touchscreen.

FIGS. 15-17 show an exemplary digit designed to interact with touchscreens. In this example, the digit is a finger 3. In other examples, a similar arrangement could be used with a thumb. The touchscreen-compatible digit of FIGS. 15-17 may avoid the need for connection to a user's body, may avoid the need for a complex conductive path through the hand, may avoid the need to connect to an electrical component such as a motor, may be suitable for retrofitting to an existing non-touchscreen-compatible digit, may avoid the need to apply conductive coatings, glue or the like, and may reliably be detected by a touchscreen.

The finger 3 includes a proximal phalanx body 301 made up of pieces 301a and 301b and a distal phalanx body 302. The proximal phalanx body 301 can house other components of the proximal phalanx 31. The proximal phalanx body 301 can be connected to the knuckle 21 via the digit drive assembly 215. The proximal phalanx body 301 has toothed sockets 308a and 308b, the teeth of which engage with teeth of the output wheels 226a and 226b (shown in FIG. 14) of the drive assembly 215. The digit drive assembly 215 drives rotation of the proximal phalanx 31 of the digit 3 in the extension-flexion direction. A link arm 306 can be connected between the knuckle 21 and the distal phalanx body 302 to cause rotation of the distal phalanx 32 upon rotation of the proximal phalanx 31, as shown in FIG. 17. In this example, the link arm 306 is connected to the knuckle 21 at mount 218 and to the distal phalanx 32 at mount 307. In this construction, the link arm 306 acts as one link of a four-bar linkage having joints at 33, 34, 218 and 307. The other links can be provided by the proximal phalanx body 301 (between joints 33 and 34), the knuckle 21 (between joints 33 and 218), and the distal phalanx body (between joints 34 and 307).

The finger 3 also includes attachments 35a, 304 and 305. The attachments 35a, 304 and 305 can be configured to attach to the finger 3 at the distal phalanx 32. One or more of the attachments 35a, 304 and 305 can be conductive and can provide a conductive path from the exterior of the finger 3 to a conductive member within the hand. In the example of FIGS. 15-17, the attachment 35a is a digit pad. Digit pads can be provided at tips of digits (fingers or thumbs) and may improve the ability of the digit to grip objects and may provide a relatively soft surface to avoid damaging objects. The digit pad 35a can be made of a polymer, for example silicone. The digit pad 35a can have a conductive additive in it to render the digit pad 35a, which may otherwise be non-conductive, conductive. The conductive additive can include or be carbon, such as carbon black, graphite, graphene, or carbon nanotubes. Carbon nanotubes may be particularly useful as they tend not to mark surfaces like touchscreens. The carbon nanotubes can be single-wall carbon nanotubes (SWCNT). The carbon nanotubes may make up between about 0.1% and about 1% or the digit pad by weight, or between about 0.1% and about 0.4% of the digit pad by weight, or about 0.3% of the digit pad by weight. In one example, the carbon nanotubes can be provided in an additive that is 10% SWCNT and 90% silicone by weight. The digit pad can be between 1% and 4% additive by weight. The balance of the digit pad (between 99% and 96%) can be silicone by weight.

The attachment 304 in this example is a retainer clip that is connected to or integral with the digit pad 35a and can be inserted into the distal phalanx body 302 to retain the digit pad 35a on the distal phalanx 32. The digit pad 35a in this example is moulded over the attachment 304. The retainer clip can clip to the distal phalanx body 302. The retainer clip 304 may or may not be conductive. In the example of FIGS. 15-17, the retainer clip 304 need not be conductive. In some examples, the retainer clip 304 is made of a polymer, for example plastic.

The attachments can include a link piece that provides a link between a conductive attachment that is at the exterior of the digit, such as the pad 35a, and a conductive member within the hand. In the example of FIGS. 15-17, the attachment 305 is such a link piece. The attachment 305 can be a spring, in one example made of metal. The spring 305 can be pressed into contact with the pad 35a when the attachments are installed in the finger 3 to maintain galvanic connection with the pad 35a. The spring 305 can also be biased into contact with the conductive member of the hand. The conductive member of the hand can be located within the finger. The spring 305 can be biased into contact with the conductive member of the hand, for example the link arm 306. The spring 305 is retained in the distal phalanx body 302 in contact with both the conductive pad 35a and the conductive member, providing a conductive path from the exterior of the finger 3 to the conductive member.

In alternative examples to the one shown in FIGS. 15-17, a tip of the digit may serve as the conductive attachment and may be in contact with the conductive member in the automated hand. The tip could have conductive additive, for example one of the conductive carbon additives listed above with reference to the pad 35a. In one example, the distal phalanx 32 can be conductive to provide the conductive attachment. The distal phalanx body 302 may be made of a conductive material such as a conductive polymer, for example conductive plastic. In some examples, the conductive polymer or plastic could have carbon nanotubes embedded in it. The distal phalanx can be attached to the rest of the digit in a retrofit operation to render a previously non-touchscreen-compatible hand touchscreen compatible, with or without the use of one or more additional conductive inserts. The conductive member can be mechanically connected to the conductive distal phalanx, which may avoid the need for an additional conductive attachment (such as the link piece 305) between the two. In one example, a conductive connection at mount 307 between the distal phalanx 32 and the link arm 306 could be provided.

In some examples the digit can be made compatible with a touchscreen by a process of retrofitting the one or more conductive attachments to a digit that is not compatible with a touchscreen. In these examples, it may be advantageous to place the conductive attachment(s) in contact with an existing member of the automated hand. Automated hands that are not compatible with touchscreens may nonetheless have members within them that can, in combination with the conductive attachment(s), emulate the effect of a human touch on a touchscreen. For example, automated hands may have metal members within them that can add sufficient capacitive loads on capacitive touchscreens to register a touch. Using existing conductive members of the automated hand may avoid the need to add special conductive members within the hand, such as dedicated conductor wires or coatings. The conductive member within the hand can be a structural member of the hand. Using a structural member of the hand may avoid the need to electrically connect the conductive member(s) at the exterior of the digit to electrical components such as motors. In the example of FIGS. 15-17, the conductive member that forms part of the electrical path is the link arm 306. The link arm 306 can be made of metal, for example aluminium or steel.

The conductive path can be insulated from electrical components of the hand. For example, the conductive path can be insulated from a housing of an actuator for the digit, such as a motor. The conductive path can be insulated from the terminals of the actuator. The insulation may be by way of substantially non-conductive materials or coatings or by one or more air gaps. The conductive path may be insulated from the body of the user by a non-conductive socket used to attach the automated hand to the user's limb. In one example, the conductive path terminates at the conductive member, e.g. link arm 306. In other examples, the conductive member can be connected to another conductive member to extend the conductive path. The other conductive member may also be a structural member of the hand. For example, the link arm 306 may be in conductive contact with the knuckle 21. The knuckle 21 can be made of metal, for example aluminium.

The conductive member can be selected or designed such that, in combination with the one or more conductive attachments, it has sufficient effect on the properties measured by a touchscreen to be detected. This may be a function of the parasitic capacitance introduced when finger 3 (or other digit) touches the touchscreen. For example, the finger 3 can be designed such that the effect of the conductive attachment(s) and conductive member on the capacitance measured by a capacitive touchscreen is within the range indicative of a human touch. In other examples, the finger 3 can be designed such that the effect on one or more detection currents applied to a touchscreen surface is within the range indicative of a human touch. It has been found that touchscreens can be quite sensitive to changes in the material in the conductive path.

The finger 3 (or other digit) may be designed for use with various types of touchscreens, including capacitive touchscreens. Capacitive touchscreens include surface capacitance or projected capacitance varieties. Within the category of projected capacitance touchscreens, there are mutual capacitance and self-capacitance touchscreen technologies.

Surface capacitance touchscreens detect a touch from changes in detection currents applied to the surface of a touchscreen at different locations, typically the corners. If a member with sufficient parasitic capacitance touches the screen, the detection currents will change as charge flows into the member. The finger 3 (or other digit) may be designed contribute sufficient parasitic capacitance to register a touch on a surface capacitance touchscreen.

Self-capacitance touchscreens work by detecting changes in capacitance between an array of electrodes and ground. When a member with sufficient parasitic capacitance touches the screen, an increase in capacitance between an electrode near the touch point and ground is detected due to the addition of the member's parasitic capacitance to ground in parallel to that of the electrode's. The finger 3 (or other digit) may be designed to contribute sufficient parasitic capacitance to register a touch on a self-capacitance touchscreen.

Mutual capacitance touchscreens work by detecting changes in capacitance between pairs of electrodes in an electrode array. When a member with sufficient parasitic capacitance touches the screen, a decrease in capacitance between a pair of electrodes that intersect near the touch point is detected due to the member drawing off charge from the electrode pair to ground. The finger 3 (or other digit) may be designed contribute sufficient parasitic capacitance to register a touch on a mutual capacitance touchscreen.

As noted above, the design of the touch-sensing arrangement may be particularly well suited to be retrofit to an existing hand to render it compatible with touchscreens. An exemplary retrofitting method may involve a preliminary procedure of removing one or more parts of the non-compatible hand to make room for the conductive attachment(s). With reference to the exemplary conductive attachments 35a and 305 of FIGS. 15-17, this may involve removing an existing digit pad. Alternatively, this may involve removing the distal phalanx 32.

A method of constructing a touchscreen-compatible hand includes attaching one or more conductive members to the digit and placing the attachment(s) in contact with a conductive member of the automated hand. The conductive member can be the one or more conductive member(s) discussed above, for example the link arm 306, and the conductive attachments can be the one or more conductive attachments discussed above, for example the pad 35a and spring 305 or the conductive distal phalanx. The method can be performed as a retrofit method, in which case the preliminary procedure of removing parts from a non-compatible hand may be performed, or as part of the initial manufacture of a touchscreen-compatible hand, in which case the preliminary procedure need not be performed.

In the example in which the conductive member is the link arm 306 and the conductive attachments are the pad 35a and the spring 305, the method can include inserting the spring 305 into the distal phalanx 32 and placing part of the spring in contact with the link arm 306. Due to the resilient nature of the spring, it can be biased against the link arm 306. The pad 35a can then be attached to the distal phalanx and in contact with the spring 305. There is now provided a conductive path from the pad 35a at the exterior of the digit to the link arm 306 via the spring 305.

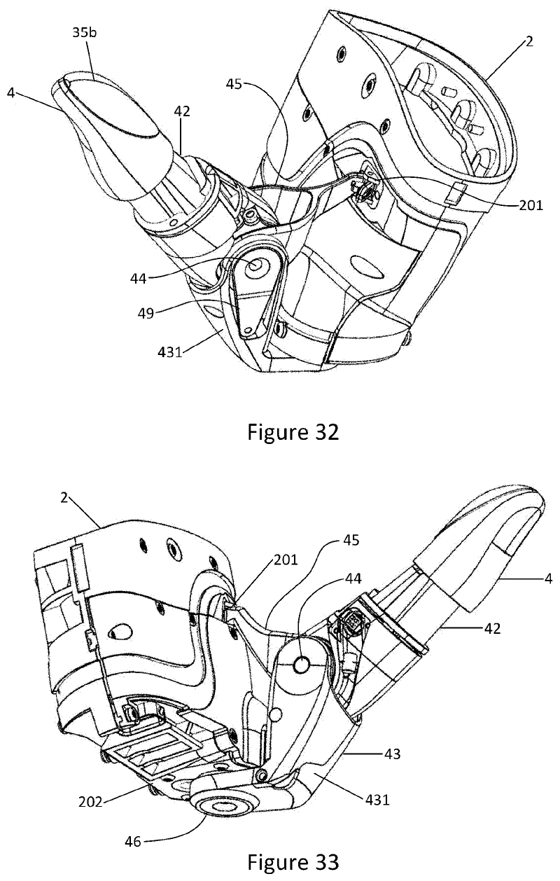

As noted above, the thumb 4 of the hand 1 may be configured for use with a touchscreen, in addition to or as an alternative to one or more of the fingers 3 being configured for use with a touchscreen. In these examples, the thumb can be provided with an attachment that contacts a conductive member in the thumb 4. One example is described with reference to FIGS. 32 to 38.

The conductive attachment(s) of the thumb can be configured to attach to distal end of the thumb, which may be a distal phalanx in a thumb that has a plurality of phalanges. One or more of the attachments can be conductive to provide a path from the exterior of the thumb 4 to a conductive member within the hand.

In the example of FIGS. 32-38, the thumb pad 35b can be a conductive attachment. The thumb pad 35b can be similar to the thumb pad 35a of FIGS. 15-17. In particular, the description of the materials, material properties, electrical properties, composition and/or construction of the finger pad 35a may also apply to the thumb pad 35b.

The thumb pad 35b can be attached to the thumb tip body 421. The thumb pad 35b can extend through the thumb tip body 421 to make contact with the thumb body 422. The thumb body 422 can be made of a conductive material, for example a conductive polymer or plastic. The thumb body 422 can be made of Nylon. The thumb body 422 can be made of a polymer or plastic with a conductive additive. The conductive additive can be carbon. The carbon can be in the form of carbon nanotubes. In alternative examples, the thumb body 422 may be made of a metal such as aluminium. The thumb body 422 can make contact with the gear housing 425, which can be made of a conductive material such as a metal, for example aluminium. The conductive path in this example can be provided by the thumb pad 35b, thumb body 422 and gear housing 425.

In the examples above, the finger pads 35a and thumb pad 35b are provided only at the ends of the digits, i.e. on the distal phalanx 32 of each finger 3 and on the thumb phalanx 42. In alternative examples, one or more additional finger pads could be provided elsewhere on the fingers 3, e.g. on the proximal phalanges 31. Similarly, one or more additional thumb pads could be provided elsewhere on the thumb 4, e.g. on the thumb metacarpal 43 and/or the auxiliary support 45. In alternative examples, a finger pad could be attached to both the distal phalanx 32 and proximal phalanx 31 of a finger and span the interphalangeal joint 34. Similarly, a thumb pad could be attached to the thumb phalanx 42 and one or both of the thumb metacarpal 43 and the auxiliary support 45.

### Cover

Automated hands may use covers to improve aesthetics by covering mechanical joints and providing a more natural-looking surface. These may also protect the joints and other workings of the hand from debris and protect foreign objects from getting caught in the joints or other workings. Covers typically cover a thumb joint and/or thumb metacarpal. These may be made of moulded elastomer material (e.g. rubber) with a concertina-like structure to allow for bending of joints. However, these look unnatural. Also, they have relatively high resistance to movement and high resilience which causes them to tend to return strongly to their neutral shape. This may make it hard to hold the position of the thumb joint without the use of non-backdrivable gears. The corrugations in the concertina structure may have a tendency to get caught in things. The moulded elastomer covers are also expensive and difficult to customise or redesign because any change would require a full retooling of the moulds for the whole cover (which is typically moulded in one piece).

Some covers may use a woven material. Woven materials can be quite stiff and resistant to initial stretching from their neutral state. Woven materials also tend to only have substantial stretch in one direction. The tight weaves of some woven materials may also prevent trapped water or other liquid from draining out through the cover. Because it can be difficult to waterproof around such a cover, water may get in behind the cover and be unable to drain out.

Some fabric covers may be unable to hold their shape well, resulting in the cover folding or sagging under its own weight when it is not pulled taut. This may result in an unnatural look and feel and may also increase the chance of the cover being caught in something.

FIGS. 18 and 19 shown an exemplary cover 41 that can cover part of the automated hand. The cover 41 includes a cover body 411. The body 411 partly covers the automated hand in use.

The body 411 can be made up of one or more regions. In the example of FIGS. 18 and 19, the body 411 has a palm region 412 that at least partly covers the palm of the automated hand in use and a thumb region 413 that at least partly covers a thumb of the automated hand in use, including covering the joint between the thumb and the palm. One or more of the regions of the cover can be made of a fabric material. Each region can be made of knitted fabric or woven fabric and can be knitted or woven from elastic threads. In one example, the palm region 412 and thumb region 413 are made of knitted material. The knitted material may be knitted with a rib stitch such as a full needle rib stitch, a Jersey stitch, or another suitable type or stitch or combination of stitches. An advantage of knitted material is that it initially has a low resistance to stretching for small amounts of stretch, but the resistance is higher for larger amounts of stretch. This may allow the cover 41 to stretch relatively easily through much or all of the range of motion of the thumb without strongly forcing the thumb back towards a neutral position and loading the thumb actuator. This may avoid the need for a non-backdrivable gearing in the thumb. The resistance to stretch can increase sharply towards higher amounts of stretch. This may help to prevent the cover 41 from being overstretched and distended.

The fabric material of the cover body 411 can be made to allow water to flow through the material due to gravity alone. This allows any water that may get behind the cover 41 to naturally drain from the automated hand. This may be achieved by using a coarse knit or weave in the fabric.

The different regions of the cover body 411 can have different properties from each other. For example, they could have different coarsenesses, different fabric constructions (e.g. knitted or woven), different stretch characteristics, different knit/weave axes, or different thread types. For example, the thumb region 413 can be made a less stretchy fabric than the palm region 412. The fabric of the palm region 412 can have a more dense (i.e. less coarse) stitch than the fabric of the thumb region 413. In one example, the thumb region 413 are both knitted. In another example, the thumb region is made of a knitted material and the palm region 412 is made of a woven material.

In some examples, the cover body 411 can be made of a polyester fibre, a polyethylene fibre such as ultra-high molecular weight polyethylene (UHMWPE) fibre, fibreglass, nylon fibre, spandex fibre, or combinations thereof. In some examples, the thumb region 413 is made from a combination of polyester fibre, UHMWPE fibre, and fibreglass and the palm region 412 is made from a combination of nylon fibre and spandex fibre. In other examples, the thumb region 413 and the palm region are both made from polyester fibre.

The cover 41 in FIGS. 18 and 19 includes seams 417a, 417b and 417c. The cover 41 in this example can be assembled from flat panels of fabric joined together at seams 417a and 417b. The seams 417a-417c can be formed using bonding tape. The tape may be made of thermoplastic polyurethane film. Alternatively or additionally, the seams 417a-417c can be sewn, knitted and/or glued. In other examples, the cover can be knitted in one piece in a 3D knitting process.

The fabric of the cover 41 can be designed with different stretch characteristics in different directions. This may help it to maintain its shape, which approximates the part of a natural human hand corresponding to the part of the automated hand that it covers. Selecting appropriate levels of stretch in each direction may help to prevent parts of the hand from sagging or folding and presenting a corrugated surface, allowing the surface of the cover to stay smooth despite movement of the thumb.

The cover 41 can have additional bracing on the cover body 411 to help maintain the shape of the cover 41. This may support it against sagging, folding, or otherwise collapsing under its own weight due to gravity. The bracing can be made of brace pieces that are more rigid than the fabric of the body. The brace pieces can encircle part of the cover body 411 to form a structural hoop, either at an edge of the body 411 or inward from the edge. The bracing can be made of polymer, such as plastic. In some examples, the bracing can be made of pieces of moulded plastic. These are shown at 414, 415 and 416 in the example of FIGS. 18 and 19. In this example, the brace 414 is provided at the edge of the cover 41 that couples to the palm. The brace 414 in this example also acts a hand coupling at which the cover 41 can be coupled to the palm of the hand. The hand coupling/brace 414 can be coupled to the hand by being sandwiched between shell pieces of the automated hand, for example between the palm cover 28 and faceplate 25 (shown in FIG. 3). The brace 415 also acts as a thumb coupling at which the thumb region 413 can be coupled to the thumb of the automated hand.

The brace pieces can be attached to the cover body 411 in various ways, for example they could be taped and/or moulded onto the cover body 411. In one example, the brace 414 (optionally made up of pieces 414a, 414b, 414c, 414d) at the edge of the cover that couples to the palm can be taped to the cover body. In one example, the braces 415 and/or 416 can be overmoulded onto the cover body 411. Specifically, the brace 415 can be overmoulded onto the fabric at the distal edge of the thumb region 413 and the brace 416 can be overmoulded onto the fabric at the proximal edge of the palm region 412.

The cover 41 can cover a wrist joint that connects the automated hand to an arm coupling. In the example of FIGS. 18 and 19, the palm region 412 can extend down over wrist joint to couple to the wrist. In this case, the brace 416 also acts as a wrist coupling and is retained in a groove (labelled 59 in FIG. 24) that encircles the wrist.

In some examples, the cover 41 could extend over the knuckles and articulated joints between the fingers and the palm. In some examples, the cover could extend back beyond the wrist to cover the connection of the wrist to a user's forearm. In other examples, the cover 41 could cover less of the palm and thumb than the example of FIGS. 18 and 19, for example covering the joint between the palm and thumb and little else.

As noted above, the braces 414, 415, 416 can encircle parts of the cover body 411 to form hoops. The hoops can be continuous or broken. In one example, the hand coupling/brace 414 is broken into pieces. This may assist in pulling the cover 41 over the hand when attaching or removing it. As best shown in FIG. 18, the hand coupling/brace 414 in this example is broken into four pieces, 414a, 414b, 414c and 414d.

The cover 41 may also have reinforced sections positioned to lie over protrusions or user input features of the hand, including over wrist release buttons or the like. The cover 41 could also have visual indicators in these locations such as differently coloured patches or symbols.

### Wrist

Some automated hands have wrist components permanently or semi-permanently attached to the palm of the hand. This may require a user to remove the entire prosthesis when the hand needs to be removed, for example for servicing.

Some wrists for automated hands have springs placed about the wrist joint for returning the joint to a neutral angle. These can be quite bulky due to the placement of springs outside of the wrist joint. Spring-based wrist joints can also be difficult to assemble because they require the springs to be pre-loaded (i.e. compressed) during assembly. These designs can also be quite heavy due to the added weight of the metal springs. Other wrists may use elastomers to return the joint to a neutral angle. Current designs using elastomers may provide only weak return forces near the neutral angle, which may prevent the wrist from returning fully to the neutral position and may make the wrist too loose or “floppy” under no load or low loads on the wrist.

Some automated hands connect to electrical components such as batteries on the user's arm using a cable that passes through the wrist. This can be difficult to connect and disconnect. Some automated hands have plug-type electrical connectors between the wrist and the hand. In these designs, a dedicated electrical release mechanism may need to be operated to release the electrical connection, in addition to the mechanical release required to decouple the hand from the wrist.

Some wrists for automated hands can be selectively locked against rotation about the flexion-extension axis. These may have locking features on the hand itself and locking buttons located in the region of the hand. Some locking buttons and the like can be difficult to operate, especially if the locking elements are not perfectly aligned.

Some wrists may screw or bolt directly to the hand. This may require space within the hand to be dedicated to screw or bolt holes.

FIG. 20 shows a wrist 5 connected to the palm chassis 26 of an automated hand. The wrist 5 in this example differs from the wrist 5′ shown in FIGS. 1-3 in that it is not a quick-disconnect wrist. By omitting quick-disconnect features from the wrist 5, the total length of the wrist 5 can be reduced.

The wrist 5 includes first coupling for coupling to a user's arm and a second coupling for coupling to the automated hand, with a wrist joint in between the couplings. In this example, the first coupling is a socket coupling 51 that couples to an arm socket on the user's remaining arm portion. This is connected to the joint body 52 which, together with the hand coupling 53, forms a wrist joint. The wrist joint in this example is articulated so that the hand coupling 53 can rotate in the flexion-extension direction at the joint. The joint body 52 is rotatably connected to the socket coupling 51 in this example, allowing the joint body 52 to rotate about the longitudinal axis of the arm socket, i.e. for pronation and supination of the hand. The wrist 5 can be selectively locked against flexion and extension at the wrist joint by the wrist lock 55.

Also shown in FIG. 20 is a cable 54a, which can electrically connect the automated hand to electrical components in the arm socket such as one or more batteries. The cable 54a can connect to the socket cable 54b, which extends from the arm socket. The cable 54 ends in an electrical connector 56 which can connect to a corresponding connector (not shown) in the automated hand. The electrical connector 56 is designed to electrically connect to, and disconnect from, the corresponding connector on the automated hand when the wrist 5 is mechanically coupled to, and decoupled from, the automated hand. This may simplify the procedure of attaching and detaching the automated hand and may ensure that the electrical connection is maintained while the hand is attached to the wrist 5.

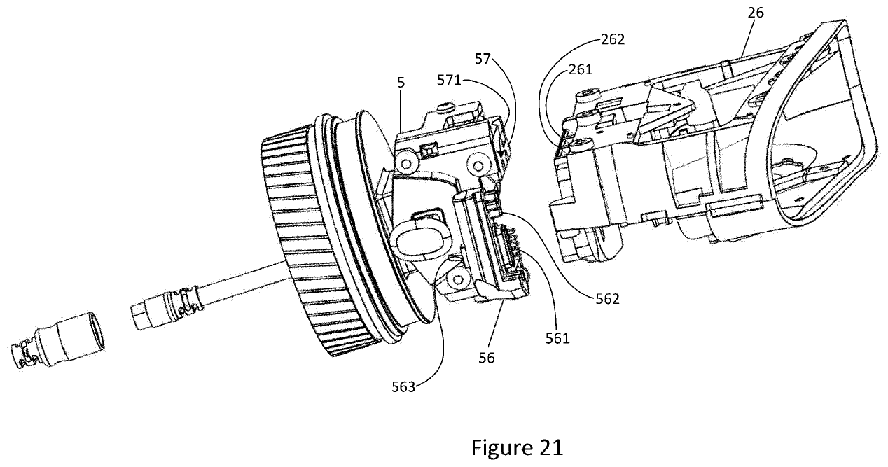

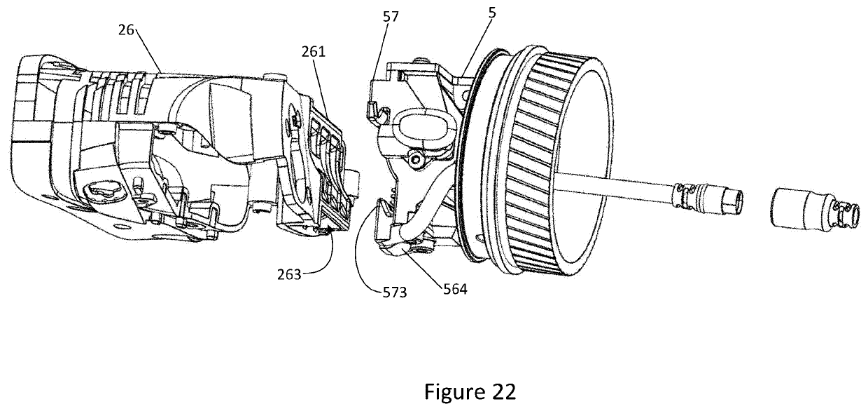

In FIGS. 21 and 22, the palm chassis 26 has been separated from the wrist 5, showing the electrical and mechanical arrangements for electrically and mechanically connecting the wrist 5 and the hand (by way of the chassis 26, in this example).

In the example of FIGS. 21 and 22, the electrical connector 56 on the wrist side has electrical terminals 561 that are biased towards complementary terminals (not shown) on the hand. The electrical terminals 561 can be spring-loaded to naturally press against the terminals on the hand when the wrist 5 and hand are brought together. In one example, the terminals 561 are pogo pins. The complementary terminals can be pads, for example flat or concave pads. In an alternative example, one or more of the biased electrical terminals are provided on the automated hand, with the complementary terminal(s) being on the electrical connector 56.

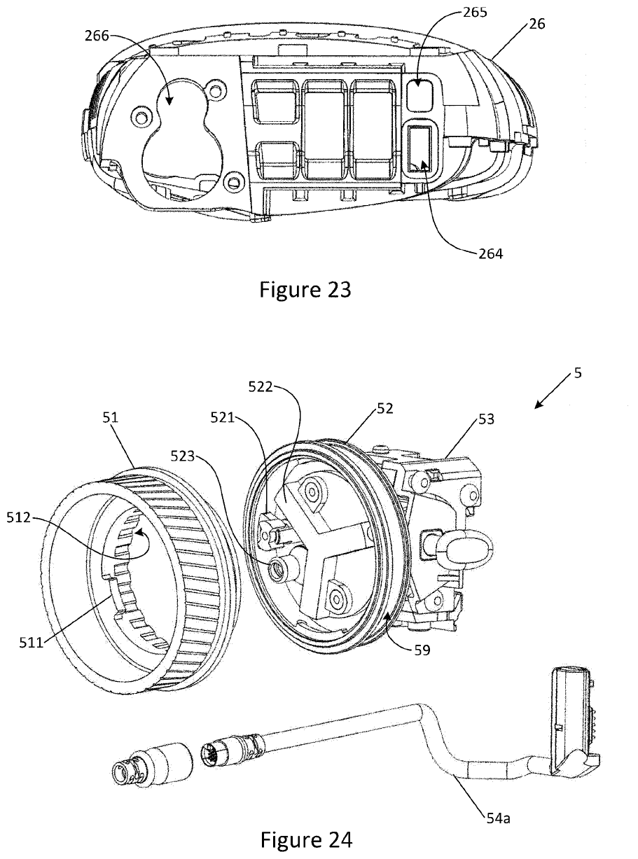

A friction fit feature can also be provided on each of the electrical connector 56 and the part of the automated hand that the wrist connects to (in this example, on the palm chassis 26). In the example of FIGS. 21 and 22, a projection 572 is formed on the electrical connector 56 that fits into an aperture (labelled 265 in FIG. 23) formed in the palm chassis 26.

A brace 563 is provided on the wrist 5 to bear on the back of the electrical connector 56 and brace it in place on the electrical connector of the hand when the wrist 5 is mechanically coupled to the hand. This may prevent the electrical coupling between the wrist and the hand being broken while the wrist 5 and hand are mechanically coupled.

The mechanical coupling between the wrist and the hand uses a clamping arrangement. In the example of FIGS. 21 and 22, the wrist 5 mechanically couples to the palm chassis 26 of the hand, but the wrist 5 may be used with hands having different structures and that couple to the wrist via different structural members. The clamping arrangement includes a coupling tongue 261 and a coupling clamp 57 that clamps to it. In this example, the coupling tongue 261 is on the hand and the coupling clamp 57 is on the wrist, but this arrangement could be reversed. The coupling tongue 261 at least partly widens out from a relatively narrow proximal portion to a wider distal portion.

The side faces of the coupling clamp 57 can be place in contact with complementary faces on the coupling tongue to restrict lateral movement between the coupling tongue 261 and the coupling clamp 57. The coupling clamp 57 can fit snugly within one or more pockets 262 and 263 of the coupling tongue 261, with the sides of the pocket providing the complementary faces that restrict movement. The pockets 262 and 263 can have ribs within them that fit snugly within slots 571 formed in the coupling clamp, also providing complementary faces that restrict lateral movement. The outward-facing sides of the pockets 262 and 263 engage with the inward-facing sides of the clamp 57. These provide the contact interface between the coupling clamp 57 and the coupling tongue 261 when clamped together. This interface is at an oblique angle to the wrist-hand longitudinal axis and causes the wrist 5 and palm chassis 26 to be pulled together when the coupling clamp 57 is tightened onto the coupling tongue 261.

The coupling clamp 57 can include a clamp plate 573. The clamp plate 573 is movable with respect to the rest of the clamp 57 to tighten and loosen. The clamp plate 573 can have screws passing through it that can be tightened and loosened to tighten and loosen the clamp plate 573, and therefore the coupling clamp 57, on the coupling tongue 261.

Also shown in FIG. 22 is a cable stay 564 on wrist 5 that helps to hold the cable in place during and after attachment of the hand to the wrist 5.

FIG. 23 shows the base of the palm chassis 26, which the wrist 5 couples to in the example of FIGS. 21 and 22. Aperture 264 would allow the terminals 561 of the electrical connector 56 to access the complementary terminals (not shown) within the automated hand. The aperture 265 is the friction fit feature that receives the projection 562 of the electrical connector 56. The aperture 266 is for a thumb mount that mounts a thumb of the automated hand to the palm chassis.

FIG. 24 shows the wrist 5 with the socket coupling 51 and cable 54a separated from the joint body 52 and hand coupling 53. As can be seen in this figure, the joint body 52 has a port 523 for the cable 54a to pass through. The joint body 52 also has detent member housings 522 that house biased detent members that engage with recess 512 in the socket coupling member. A rotation stop 521 is also provided on the joint body 52 to engage with rotation stop 511 of the socket coupling 51 to limit rotation of the wrist in the pronation-supination direction.

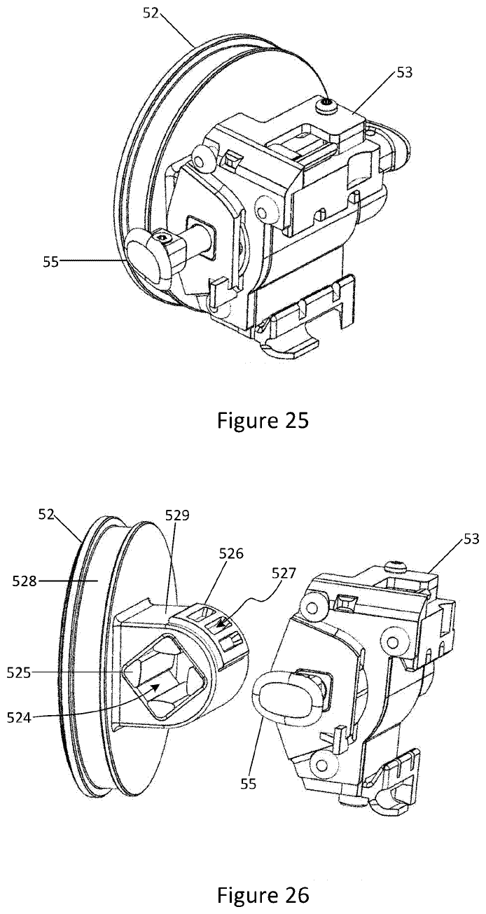

FIG. 25 shows the joint body 52 and hand coupling 53, which together form a wrist joint. In other examples, the joint body 52 could be connected to an intermediate body that is between the joint body 52 and the hand coupling to form a wrist joint.

In the example of FIG. 25, the wrist joint is rotatable in the flexion-extension direction. A wrist lock 55 is also shown, which can be operated to selectively lock the wrist joint to restrict flexion and extension.

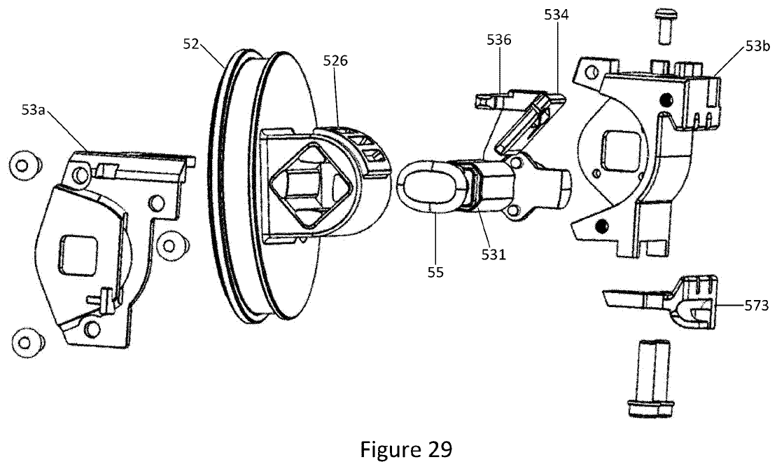

As shown in FIG. 26, the joint body 52 has a rigid mounting post 529 that the hand coupling 53 is rotatably mounted to. The mounting post 529 has an aperture 524 extending through it. The mounting post 529 defines a rigid sleeve surrounding the aperture 524. The sleeve, and hence the aperture 524, is non-circular. A rigid axle (shown as 531 in FIG. 29) can extend through the aperture 524. The non-circular aperture can be generally polygonal in cross section. In one example, the non-circular aperture is generally square. The rigid axle can have the same general shape as the aperture 524 in cross section, for example it can also be square. In a neutral orientation, there can be an angular offset between the aperture 524 and the axle such that corners of the axle are located at the midpoints of sides of the aperture 524.

Resiliently deformable pieces 525 are located in the aperture 524 and go between the sleeve and the rigid axle. When the rigid axle rotates within the aperture, the resiliently deformable pieces 525 are deformed. Due to their resilience, they act to force the rigid axle back towards a neutral orientation in which forces on the axle are balanced. The resiliently deformable pieces 525 can be made of elastomers or suitable polymers. For example, the resiliently deformable pieces 525 could be made of silicone or rubber. The resiliently deformable pieces 525 can be located in the corners of the aperture 524 and can be shaped to fit within the space between the rigid sleeve and the rigid axle in the neutral axle orientation.

Also shown in FIG. 26 is lock base 526 for the locking mechanism. The lock base 526 includes recesses 527. As will be detailed further with reference to FIGS. 28-30, the recesses 527 can receive a locking plate of the locking mechanism. The recesses can be arranged radially around the rotational axis of the wrist joint. The locking plate 534 can move towards and away from the rotational axis of the wrist joint to lock and unlock the wrist.

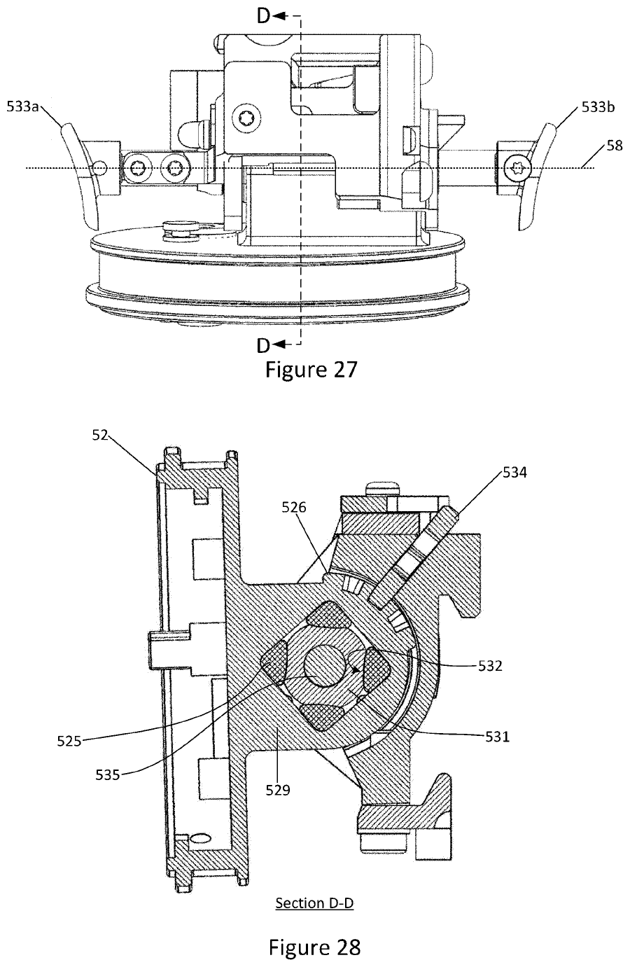

FIG. 27 shows the wrist joint from above. The flexion-extension rotational axis of the wrist joint is indicated at 58. FIG. 28 is a cross sectional view taken along the line D-D.

In FIG. 28, the rigid axle 531 is shown within rigid sleeve provided by the mounting post 529. The resiliently deformable pieces 525 are shown between the rigid sleeve and the rigid axle 531. The wrist joint is shown in neutral orientation to which the resiliently deformable pieces act to return the wrist joint.

In the example of FIG. 28, the rigid axle 531 has at least one non-planar face 532. It has been found that the propensity of the wrist joint to return accurately to the neutral orientation can be controlled by shaping the faces of the rigid axle 531. The non-planar face 532 in this example is concave. Concave faces have been found to provide good return torques towards the neutral orientation in the case of small deviations from the neutral orientation. They can also accurately return the wrist joint to the neutral orientation. In this example, all four faces of the square rigid axle 531 are concave. In some examples, planar faces may be used in addition to or instead of non-planar faces 532.

A lock rod 535 that forms part of the locking mechanism is also shown. In this example, the lock rod 535 passes through the rigid axle 531 and extends along the wrist joint rotational axis 58. The wrist lock 55 can be operated by pushing the buttons 533a and 533b along the axis 58. This may make operation of the wrist lock 55 easier for a user than placing the wrist lock in another location such as on the hand coupling 53 or on the hand itself, because in those cases the wrist lock actuator (e.g. button or the like) will move when the wrist rotates, rather than always remaining in one place. It may also be particularly intuitive for a user to lock rotation of the wrist joint by pushing the buttons 533a, 533b along the wrist joint rotation axis 58.

The locking mechanism also includes a locking member which engages with the lock base 526. In this example, the locking member is locking plate 534. The locking plate 534 is shown inserted into one of the recesses, in this case the recess that corresponds to the neutral position.

An alternative wrist joint is shown in FIG. 39. In this example, the resiliently deformable pieces 525′ can be approximately circular in cross section in the neutral state. The non-circular axle 531′ can be approximately in the shape of a circle in cross section except for recesses 532′ for receiving the resiliently deformable pieces 525′. In particular, the perimeter of the axle 531′ can include part-circular arcs 531′a between the recesses. The recesses 532′ can be part-circular, for example approximately or somewhat less than half-circular. Alternatively, the recesses 532′ can be planar. The non-circular aperture in the rigid sleeve, which is defined by the mounting post 529′, can be approximately in the shape of a circle except for recesses 524′b in which the resiliently deformable pieces 525′ are located. Part-circular arcs 524′a can be provided between the recesses 524′b.

The recesses 532′ and 524′b are recessed in the sense that they correspond to parts of the perimeter of the rigid axle 531′ and aperture in the rigid sleeve that are recessed from hypothetical circles on which the respective part-circular arcs lie. In other words, the recesses 532′ of the rigid axle 531′ are formed within the perimeter of a hypothetical circle on which the part-circular arcs 531′a of the rigid axle 531′ lie. The recesses 524′b of the aperture in the rigid sleeve are formed outside of the perimeter of a hypothetical circle on which the part-circular arcs 524′a of the aperture lie.

The recesses 524′b can be part-circular, for example approximately or somewhat less than half-circular. As shown in FIG. 39, each of the resiliently deformable pieces 525′ can be held between respective recesses 532′, 524′b of the axle 531′ and the rigid sleeve. The resiliently deformable pieces 525, 525′ can be placed at a plurality of angular positions around the axis of rotation of the wrist joint. The resiliently deformable pieces can be approximately evenly spaced around the axis of rotation. In each of the examples of FIGS. 26-29 and 39 there are four resiliently deformable pieces 525, 525′, equally spaced around the axis of rotation. In alternative examples, there may be more or fewer than four resiliently deformable pieces. For example, there may be one, two, three, or more than four resiliently deformable pieces 525, 525′.

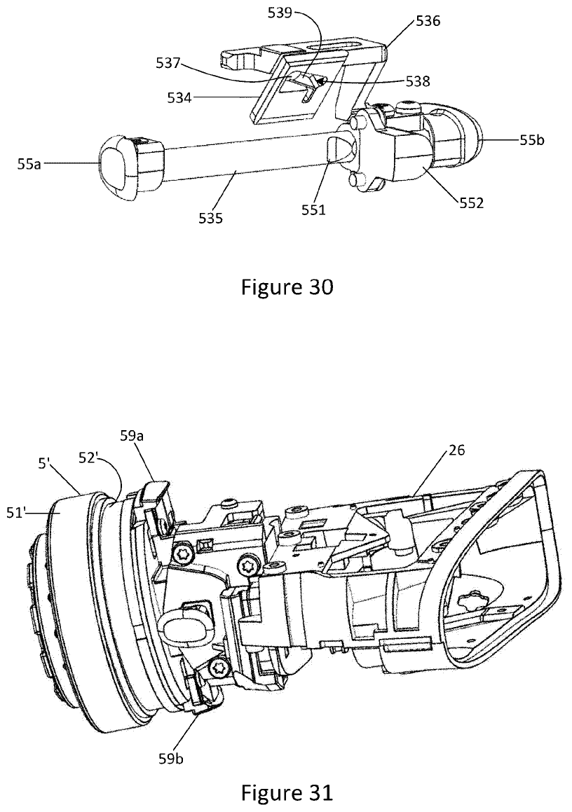

The locking mechanism is shown in more detail in FIGS. 29 and 30. In FIG. 29, the joint body 52 has been separated from the hand coupling 53 and the wrist lock 55. The hand coupling 53 has been exploded into housing bodies 53a and 53b and clamp plate 573. The wrist lock 55 is shown with the rigid axle 531 and includes the locking plate 534 and carrier 536.

FIG. 30 shows the locking mechanism in isolation. The locking buttons 533a and 533b are attached to the rod 535. The carrier 536 is attached to the rod 535. A pin 537 protrudes from the carrier 536 into the recess 538 in the locking plate 534. Within the recess 538 is a spring 539. The spring may be a flat spring. The spring may be V-shaped and of a type that is sometimes known as a V-spring. The locking buttons 533a and 533b, rod 535 and carrier 536 constitute a locking actuator is operated to lock and unlock the wrist. In this example, the wrist is locked by a user manually pushing the buttons 533a, 533b. In other examples, a powered actuator such as an electric or electromagnetic actuator could be provided.

The rod 535 has recesses 551 on it. These can be engaged with by a detent member in the detent housing 552. For example, the detent housing may include a spring and a ball detent biased towards the rod 535 by the spring.

To operate the wrist lock, a user operates the locking actuator—for example by pressing the button 533b to lock the wrist. The pin 537 will move from right to left (in the orientation of FIG. 30) within the recess 538. If the locking plate is aligned with a recess 527 of the lock base 526, the pin 537 will ride over the sloped surface of the spring 539, pushing it downwards to drive the locking plate 534 down into the recess 527. If the locking plate 534 is not aligned with a recess 527, when the pin rides over the sloped surface of the spring it will compress the spring 539. When the wrist joint rotates such that the locking plate 534 is aligned with a recess 527 of the lock base 526, the spring will resile to its normal shape and drive the locking plate 534 into the recess 527.

To unlock the wrist, a locking actuator is operated in the opposite direction (e.g. by pushing button 533a). The pin 537 the rides over the sloped surface of the recess 538 and drives the locking plate up out of the recess 527 in the lock base 526.

This mechanism may ensure that the locking actuator is easy to actuate (e.g. the buttons 533a, 533b are easy to push) even when the locking plate 534 is not aligned with a recess 527 of the lock base 526.

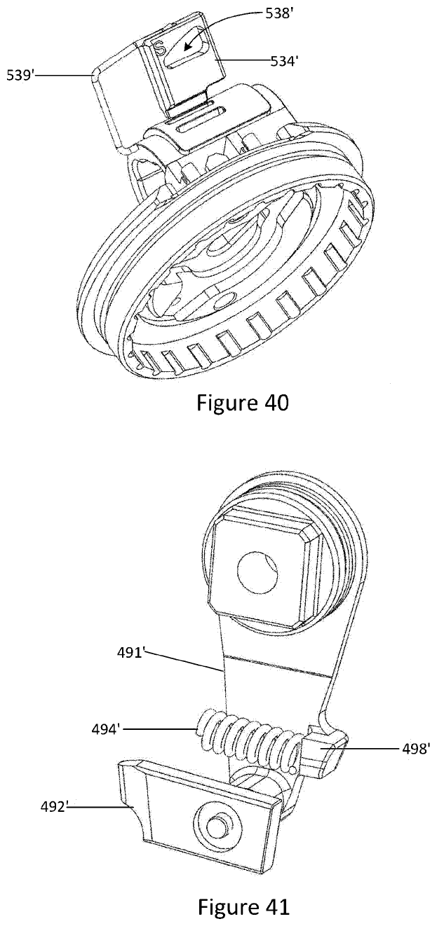

FIG. 40 shows components of an alternative locking mechanism. In this example, the locking mechanism is similar to the one of FIGS. 25-30 but with a different spring. In FIG. 40, an external spring 539′ provides a bias against the outside of the plate 534′. This removes the need for a spring in the recess 538′. In this example, the spring 539′ is a spring wireform. The spring 539′ can be a non-coiled spring.

FIG. 31 shows an alternative form of wrist 5′ coupled to the palm chassis 26. This wrist 5′ is also shown in FIGS. 1-3. This wrist 5′ is a quick disconnect wrist. By squeezing the 59a and 59b, a user can quickly decouple the socket coupling 51′ from the joint body 52′. The wrist 5′ can be the same as the wrist 5 in other respects. In some examples, the wrist 5′ can be provided with quick-disconnect features as set out in Patent Publication No. WO2021/177840A1 to 5^{th} Element Limited.

In another example, a further alternative wrist may be provided that does not have a wrist joint allowing for flexion and extension in the wrist. This may save space in the wrist, resulting in a shorter wrist. Due to the omission of the wrist joint, a single body can serve as the hand coupling and joint body and be rotatably connected to the socket coupling. This wrist may be similar to the wrist 5 in other respects.

### Thumb

Some automated hands have digits that correspond to fingers and thumbs of natural human hands. The thumbs may be able to rotate in an antepositioning-retropositioning direction to move in and out of opposition with the fingers. When executing certain grips, it can be important to ensure the thumb is accurately positioned and does not rotate undesirably in the antepositioning-retropositioning direction. For this reason, some automated hands may include a thumb lock for preventing this rotation. Some thumb locks may lock a thumb against rotation in the antepositioning-retropositioning direction when it has rotated to a certain point, for example into opposition with the fingers. These automated hands are limited to only a small number of different positions at which the thumb can be locked. Also, the locking of the thumb is not in response to a function performed by the thumb that would require locking, like executing a grip. Other hands may use engagement surfaces located on the back of the thumb and on a locking member located behind the thumb. These restrict rotation of the thumb when the thumb is pushed back onto the locking member to bring the engagement surfaces together. It may be desirable to provide an automated hand that doesn't require a locking member behind the thumb.

Some automated hands include a thumb that is mounted to the palm only at the base of the thumb. When a force is applied to the thumb, producing a torque at the connection point. The connection point has to withstand this torque itself and counteract the entire applied force. The closer to the base of the palm the thumb is connected, the longer the lever arm provided by the thumb is and the greater the torque at the connection point is for a given applied force. It may be desirable to provide an additional support to the thumb that resists a component of the applied force, may improve the resistance of the thumb to breaking, and may allow the thumb connection to be lower on the palm than it otherwise could be. Moving the thumb connection point lower down on the palm may save space in the palm.

Some automated hands include a thumb that is substantially non-compliant but that is attached to an actuator that is compliantly mounted in the palm. It may be advantageous to separate the compliance of the thumb from the mounting to the palm. This may improve sealing of the palm, improve structural strength of the thumb mount, and reduce the space needed in the palm for the thumb mount.

FIGS. 32-34 show a thumb 4 according to one example. The thumb 4 is connected to a palm 2 and forms part of an automated hand. For clarity, only part of the palm 2 is depicted in these figures. The thumb 4 is pivotally mounted to the palm 2 at the connection 46. The connection 46 can be low down on the palm, for example at or near the base 202 of the palm 2. Mounting the thumb 4 to the base 202 of the palm 2 may save space in the palm 2.

Also shown in FIG. 32 is the thumb pad 35b. As detailed in the “Touchscreen Compatibility” section above, the thumb pad 35b may be conductive attachment configured to make the thumb compatible with a touchscreen.

An auxiliary support 45 provides an additional structural connection between the thumb 4 to the palm 2. The auxiliary support 45 may help to support the thumb 4 and reduce torque on the connection 46 at base of the thumb 4 when the thumb 4 is forced back.

The auxiliary support 45 extends from an intermediate location along the thumb 4 to a connection point 201 on the palm 2. The intermediate location on the thumb is placed between the ends of the thumb 4, for example in from either end by at least 10% of the length of the thumb, or by at least 25% of the length of the thumb 4. The auxiliary support 45 can connect to the thumb at or near an articulated joint 44 between two segments of the thumb 4. In the example of FIGS. 32 and 33, the auxiliary support 45 connects to the thumb 4 at the articulated joint 44 between the thumb metacarpal 43 and the thumb phalanx 42. In other examples, the auxiliary support 45 could connect to the thumb 4 distal of the articulated joint 44 or proximal of the articulated joint 44.

The connection point 201 on the palm 2 is spaced apart from the connection 46 to which the base of the thumb 4 is connected. This allows the auxiliary support 45 to act on the thumb 4 along a line that is largely tangential to an arc swept out by the thumb when it is forced back under load. The connection point 201 on the palm 2 is between the base of the palm 2 and the distal end of the palm 2 (where the fingers attach).

The auxiliary support 45 can be flexible. The auxiliary support 45 can bend or extend to allow the thumb 4 to comply under loads. In some examples, the auxiliary support 45 can be made of nylon. The auxiliary support 45 of FIGS. 32 and 33 is a support arm. The support arm 45 can be made of a resilient material such as a polymer. In some examples, the support arm can be made of plastic or nylon. The support arm 45 is sufficiently rigid to hold its shape when the thumb is not under load but flexible enough to comply and allow the thumb to move somewhat when the thumb is under load. The resilience of the support arm 45 is sufficient to return it to its default shape after the load is removed.

The connection 201 of the support arm to the palm can be a pivotal connection, allowing the support arm to pivot with little resistance when the thumb pivots in the antepositioning or retropositioning directions. Alternatively, the support arm could be sufficiently flexible to bend to accommodate pivoting of the thumb 4. The support arm 45 can connect to the thumb 4 at both sides for a secure connection on the thumb. This may also avoid twisting the thumb 4 about its longitudinal axis when load is applied. To enable this, the support arm 45 can be forked at the thumb end. The support arm 45 can be curved so that it can straighten out under tension, allowing compliance to the thumb 4 to a load. The “upper” side of the support arm 45, i.e. the side facing away from the base of the palm 2, can have a concave portion to assist gripping of objects. The support arm 45 can be arranged so that it does not interfere with the palm or thumb anywhere across their full range of motion.

In an alternative example, the auxiliary support could be a cord.

The thumb 4 can have a compliant portion between its connection 46 to the base 202 of the palm 2 and the intermediate portion at which the auxiliary support 45 connects to allow the thumb 4 to move back under load. In the example of FIGS. 32 and 33, the compliant portion is the metacarpal housing 431. Providing a compliant portion on the thumb 4 means that the mounting of the thumb 4 need not be compliant. The pivotal connection 46 of the thumb 4 to the palm 2 can include a rigid mount. A rigid mount will move less that a compliant mount or substantially not at all during operation of the hand, which may make it easier to waterproof the hand at this point.

The auxiliary support 45 may be frangible such that it can reliably break under a certain tension. The pivotal connection of the thumb 4 to the palm 2 can be a safety pivot that allows the thumb to pivot freely in the extension direction when the auxiliary support 45 breaks.

A catch 49 is also provided to restrict pivoting of the thumb 4 in the antepositioning-retropositioning directions when the thumb 4 is under load. This will be described in more detail with reference to FIGS. 35-38.

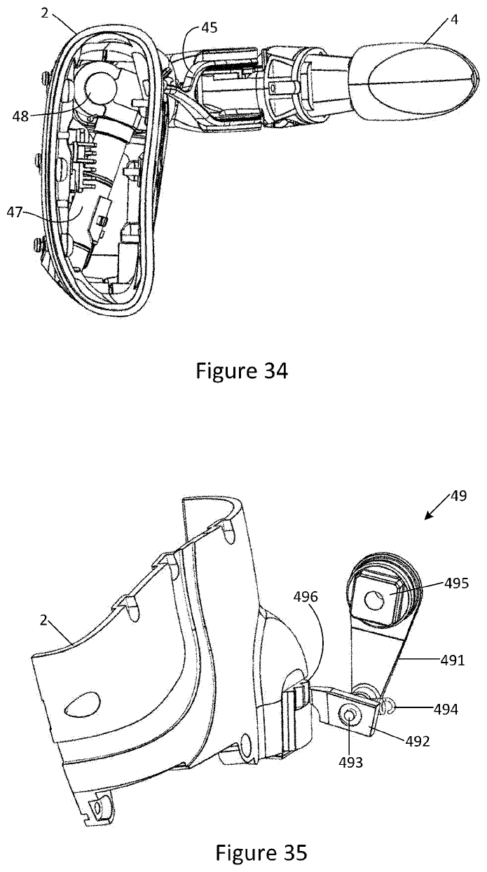

FIG. 34 shows the palm 2 and thumb 4 from a top elevation. Within the palm is shown a thumb actuator 47 that causes pivoting of the thumb in antepositioning and retropositioning. The thumb actuator 47 can be motor, e.g. a BLDC motor. The thumb actuator 47 can drive the thumbs pivoting via one or more gears in the gearbox 48. The fork in the support arm 45 can also be seen in FIG. 34.

FIG. 35 shows the catch 49 in more detail. The catch 49 includes a tooth 492 that can engage with one of the recesses 496 on the palm to lock the thumb against pivoting. The catch 49 also includes a spring 494 to bias the tooth 492 away from the recesses 496. The tooth 492 is linked to the link arm 491, which has an axle 495 on it. The axle 495 is configured to engage with a compliant portion of the thumb that complies under load on the thumb 4, thereby moving link arm 491 and driving tooth 492 into a recess 496.

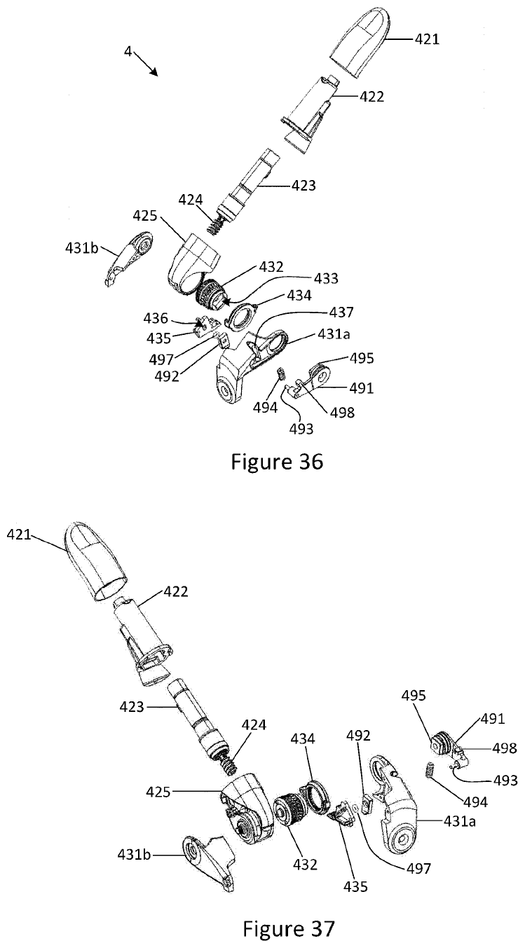

Further details of the catch 49 and thumb 4 are shown in the exploded views of FIGS. 36 and 37. The thumb 4 includes a thumb flexion-extension actuator 423. This actuator 423 can be a motor, e.g. a BLDC motor. The motor 423 is located within thumb body 422, which is in turn partly located within the thumb tip body 421.

Attached to the motor is a motor-driven gear 424 which drives the gear 432 which is located in the gear housing 425. The gear 432 is compliantly mounted so that it can comply under applied loads. In particular, the gear 432 can rotate somewhat within the housing 425. The bushing 434 is placed over the gear 432 and the gear 432 is retained between the metacarpal housing parts 431a, 431b. In this example, the motor-driven gear 424 is a worm and the gear 432 is a worm wheel. When assembly, the axle 495 fits within the socket 433 of the gear 432 to rotationally couple the link arm 491 to the gear 432. An end of the spring 494 is looped over the pin 493 and is housed within the recess 437 on the metacarpal mount part 431a. The pin 493 on the link arm 491 can extend through an aperture in the metacarpal housing part 431a into the tooth 492. The pin 493 is retained in place by the retainer ring 497. The end of the pin 493 extends into the aperture 436 on the block 435. This controls the limits of movement of the pin 493 and thereby the limits of extension of the tooth 492 and rotation of the gear 432. The finger 498 also controls the limit of extension of the tooth 492 by engaging with the metacarpal housing part 431a.

In operation, the actuator 423 can rotate the gear 424 which is engaged with the gear 432. The gear 432 serves as a fixed gear that the gear 424 can move around as it rotates, except that the gear 432 is not completely fixed and can rotate a small amount. When the gear 424 moves anticlockwise over the gear 432, the thumb 4 extends. When the gear 424 moves clockwise over the gear 432, the thumb 4 flexes. When the gear 424 drives the gear 432 for flexion, the gear 432 can rotate somewhat against the bias of the spring 494. This rotation of the gear 432 drives rotation of the link arm 491 and causes the tooth 492 to move into the recesses on the palm, thereby locking the thumb 4 against pivoting in the antepositioning-retropositioning direction. In an alternative example, a compression spring 494′ can be located on the palm side of the finger 498′, as shown in FIG. 41, in place of the tension spring 494 of FIGS. 35-37. This spring 494′ is compressed in use and is coupled to the link arm 491′ to bias the tooth 492′ away from the recesses on the palm. It will be appreciated that the spring 494′ only needs to be kinematically coupled to the link arm 491′—it does not need to be attached to it, although in some examples it could be attached to the link arm 491′ directly or indirectly. For example, the spring 494′ could be coupled to the link arm 491′ by being biased against the link arm 491′ or an object (such as the finger 498′) that is coupled to the link arm 491′, without the need for a direct attachment to the link arm 491′ of the object coupled to the link arm.

When the thumb 4 is forced back (i.e. towards extension), the phalanx part of the thumb 4 rotates back with respect to the metacarpal part. This causes the gear 424 in the phalanx part to push on the compliant gear 432. The compliant gear 432 complies and rotates anticlockwise, thereby rotating the link arm 491 and driving the tooth 492 to move into the recesses on the palm, locking the thumb against pivoting.

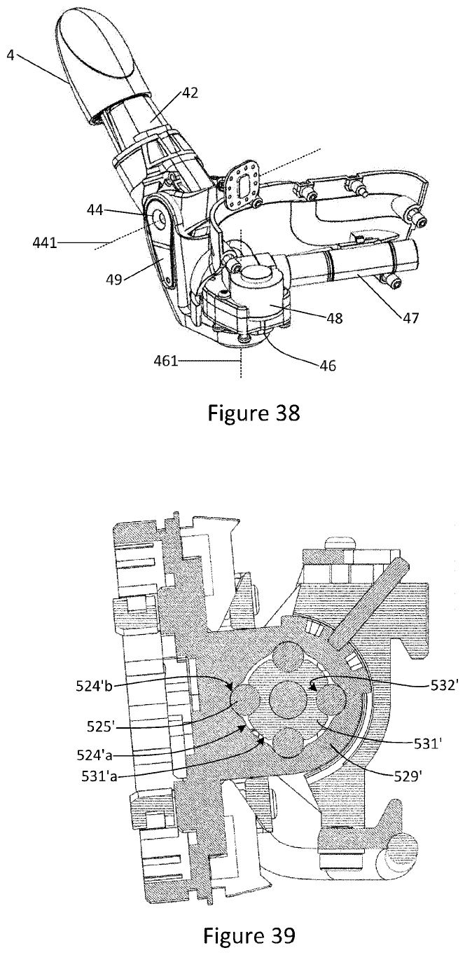

FIG. 38 illustrates the axes about which the thumb 4 can pivot and rotate. The actuator 47 drives pivoting of the entire thumb 4 about the antepositioning-retropositioning axis 461 by way of gears in the gearbox 48. This pivots the thumb 4 at its connection 46 to the base of the palm. An actuator (shown as 423 in FIG. 37) is located within the thumb phalanx 42 and drives flexion and extension of the thumb 4 at the articulated joint 44. This rotation is about the flexion extension axis 441.

With reference to the axes of FIG. 38, the catch 49 restricts pivoting of the thumb 4 about the axis 461 when the actuator in the phalanx 42 drives flexion of the thumb 4 about the axis 441 or when an external load is applied to the thumb 4 to force it towards extension about the axis 441.

The thumb catch described herein may help to maintain an accurate thumb position when executing grips. It may also keep the thumb at a fixed angle about the axis 461 when flexing the thumb to help accurately bring it together with fingers of the hand. It may also allow more precise control of the thumb angle about the axis 461. The thumb catch arrangement may also avoid the need for catches or rotation lock components at the back of the thumb, resulting in a more anatomically correct hand.

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of the Applicant's general inventive concept.

1.-226. (canceled)

227. A digit mounting arrangement for a prosthetic hand, the digit mounting arrangement comprising:
a. a rigid mount configured to be located in or on a palm of the prosthetic hand;
b. a resiliently deformable sleeve located within the rigid mount, the resiliently deformable sleeve having a sleeve aperture therein; and
c. a connector located within the sleeve aperture, the connector having a digit extending therefrom, wherein the digit is configured to be moveable relative to the palm;
the digit mounting arrangement being arranged such that the connector can rotate with respect to the mount when a force is applied to the digit extending therefrom.

228. The digit mounting arrangement of claim 227, wherein the arrangement is such that the connector can rotate in the plane of the palm.

229. The digit mounting arrangement of claim 227, wherein the arrangement is such that the connector can rotate in a plane normal to the plane of the palm.

230. The digit mounting arrangement of claim 227, wherein the arrangement is such that the connector can rotate about its longitudinal axis.

231. The digit mounting arrangement of claim 227, wherein the arrangement is such that the connector can translate with three translational degrees of freedom.

232. The digit mounting arrangement of claim 227, wherein the connector is part of a digit drive that drives flexion and/or extension of the digit.

233. The digit mounting arrangement of claim 232, wherein the connector comprises an actuator.

234. The digit mounting arrangement of claim 233, wherein the actuator comprises a motor.

235. The digit mounting arrangement of claim 232, wherein the connector is connected between an actuator and the digit.

236. The digit mounting arrangement of claim 227 comprising an actuator in the digit, the actuator driving flexion and/or extension of the digit.

237. The digit mounting arrangement of claim 227, comprising a pivot between the connector and the rigid mount, the connector being rotatable with respect to the mount about the pivot.

238. The digit mounting arrangement of claim 237, wherein the pivot comprises one or more pairs of bearing surfaces, one of the bearing surfaces being on or coupled to the rigid mount and the other being on or coupled to the connector, wherein the bearing surfaces of each pair are in close proximity to each other.

239. The digit mounting arrangement of claim 238, wherein the resiliently deformable sleeve has one or more apertures formed therein and wherein one of the bearing surfaces of each pair is on a protrusion that protrudes at least partly through a respective one of the apertures.

240. The digit mounting arrangement of claim 227 further comprising a rigid sleeve between the connector and the resiliently deformable sleeve.

241. The digit mounting arrangement of claim 240, wherein the rigid sleeve is configured to couple the connector to the rigid mount.

242. The digit mounting arrangement of claim 241, wherein the rigid sleeve comprises one or more twist-lock features to twist lock to complementary twist lock features on a retainer that retains it with respect to the rigid mount.

243. The digit mounting arrangement of claim 238 further comprising a rigid sleeve between the connector and the resiliently deformable sleeve wherein one of the bearing surfaces of each pair is provided on the rigid sleeve.

244. The digit mounting arrangement of claim 227 further comprising a seal between the connector and the rigid mount to prevent liquid from entering a sealed region within the prosthetic hand.

245. The digit mounting arrangement of claim 237 further comprising a seal between the connector and the rigid mount to prevent liquid from entering a sealed region within the prosthetic hand wherein the seal is located near the pivot.

246. The digit mounting arrangement of claim 227, wherein the rigid mount is configured to provide limits to the rotation of the connector with respect to the mount.

247. The digit mounting arrangement of claim 246, wherein an inner surface of the rigid mount within which the resiliently deformable sleeve sits is dimensioned to control the maximum lateral rotation of the connector about one or more axes.

248. The digit mounting arrangement of claim 246, wherein an inner surface of the rigid mount includes one or more rotation restraints to limit rotation of the connector about its longitudinal axis.

249. The digit mounting arrangement of claim 227, wherein the connector is coupled to the digit by an articulated joint.

250. The digit mounting arrangement of claim 227, wherein the resiliently deformable sleeve comprises an elastomer, rubber, silicone, or a polymer.

251. The digit mounting arrangement of claim 227, wherein the resiliently deformable sleeve comprises a material with a DMTA damping factor of between about 0.05 and about 0.8 over a temperature range of about −20° C. to about 100° C.

252. A prosthetic hand comprising:
a palm
a plurality of rigid mounts attached to the palm;
a plurality of resiliently deformable sleeves, each with a respective sleeve aperture therein, wherein each resiliently deformable sleeve is located within a rigid mount; and
a plurality of connectors, each located within a respective sleeve aperture, each connector having a digit extending therefrom;
wherein each connector can rotate with respect to its respective rigid mount when a force is applied to the digit extending therefrom.

253. The prosthetic hand of claim 252 comprising a plurality of rigid mounts, each resiliently deformable sleeve being located within a respective one of the plurality of rigid mounts.

254. The prosthetic hand of claim 253, wherein the rigid mounts are integral with each other.

255. A digit mounting arrangement for a prosthetic hand, the digit mounting arrangement comprising:
a rigid mount configured to be located in or on a palm of the prosthetic hand, the rigid mount having a plurality of mount apertures therein;
a plurality of resiliently deformable sleeves, each with a respective sleeve aperture therein, wherein each resiliently deformable sleeve is configured to be located within a respective one of the mount apertures; and
a plurality of connectors, each configured to be located within a respective sleeve aperture, each connector having a digit extending therefrom;
wherein each connector is configured to move with respect to the rigid mount when the connector is located within the respective sleeve aperture, the respective sleeve is within the respective mount aperture, and a force is applied to the digit extending from the connector.
